(19) United States
(12) Patent Application Publication
(10) Pub. No.: US 2012/0088791 A1
(43) Pub. Date: Apr. 12, 2012

(54) AMIDOBIPIPERIDINECARBOXYLATE M1 ALLOSTERIC AGONISTS, ANALOGS AND DERIVATIVES THEREOF, AND METHODS OF MAKING AND USING SAME

(75) Inventors: Craig W. Lindsley, Brentwood, TN (US); Jeffrey P. Corn, Brentwood, TN (US); Charles David Weaver, Franklin, TN (US); Colleen M. Niswender, Brentwood, TN (US); Evan P. Lebois, Atlanta, GA (US); Thomas M. Bridges, Nashville, TN (US)

(73) Assignee: VANDERBILT UNIVERSITY, Nashville, TN (US)

(21) Appl. No.: 13/202,272

(22) PCT Filed: Feb. 19, 2010
(86) PCT No.: PCT/US2010/024802

§ 371 Date: Nov. 16, 2011

Related U.S. Application Data
(60) Provisional application No. 61/153,964, filed on Feb. 19, 2009.
(60) Provisional application No. 61/158,105, filed on Mar. 6, 2009.
(60) Provisional application No. 61/174,961, filed on May 1, 2009.

(51) Int. Cl. A61K 31/4545; C07D 409/14; A61P 25/18; A61P 25/16; A61P 25/24; A61P 25/00; A61P 25/28; A61P 25/14; C07D 401/04; A61P 25/22

(52) U.S. Cl. 514/316; 546/189; 546/187

(57) ABSTRACT

In one aspect, the invention relates to compounds having a general structure: Formula (I) which are useful as allosteric agonists of the M₁ muscarinic receptor, synthetic methods for making the compounds; pharmaceutical compositions comprising the compounds; and methods of using the compounds, for example, in treating neurodegenerative diseases, including Alzheimer's Disease. This abstract is intended as a scanning tool for purposes of searching in the particular art and is not intended to be limiting of the present invention.

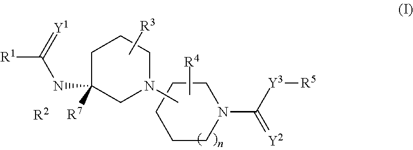

### CROSS-REFERENCE TO RELATED APPLICATIONS

This application claims the benefit of U.S. Application No. 61/153,964, filed Feb. 19, 2009; the benefit of U.S. Application No. 61/158,105, filed Mar. 6, 2009; and the benefit of U.S. Application No. 61/174,961, filed May 1, 2009; which are all hereby incorporated herein by reference in entirety.

### BACKGROUND

Alzheimer's Disease (AD) is a neurodegenerative disease affecting the elderly, which results in progressive impairment of memory, language skills and severe behavioral deficits. Hallmarks of the disease include degeneration of cholinergic neurons in the cerebral cortex, hippocampus, basal forebrain and other regions of the brain important for memory and cognition. Other hallmarks of AD include neurofibrillary tangles composed of hyperphosphorylated tau and accumulation of amyloid β peptide (Aβ). Aβ is a 39-43 amino acid peptide produced in the brain by proteolytic processing of β-amyloid precursor protein (APP) by the β-amyloid cleaving enzyme (BACE) and gamma secretase which leads to accumulation of Aβ in the brain, where Aβ 1-40 and 1-42 are the principal aggregate-forming species of Aβ.

Schizophrenia is a debilitating psychiatric disorder characterized by a combination of negative (blunted affect, withdrawal, anhedonia) and positive (paranoia, hallucinations, delusions) symptoms as well as marked cognitive deficits. While schizophrenia remains an idiopathic disorder, it appears to be produced by a complex interaction of biological, environmental, and genetic factors. Over 40 years ago it was found that phencyclidine (PCP) induces a psychotic state in humans that is very similar to that observed in schizophrenic patients. The finding that the main mode of action of PCP is that of a non-competitive antagonist of the N-methyl-D-aspartate (NMDA) subtype of ionotropic glutamate receptor stimulated a series of studies that have led to the development of the NMDA receptor hypofunction model of schizophrenia. Besides schizophrenia, dysfunction of glutamatergic pathways has been implicated in a number of disease states in the human central nervous system (CNS) including cognitive deficits, dementias, Parkinson's disease, Alzheimer's disease and bipolar disorder.

NMDA receptor function can be modulated by activation of G Protein-Coupled Receptors (GPCRs) that are known to physically and/or functionally interact with the NMDA receptor. The NMDA receptor hypofunction hypothesis is an alternative proposal to explain the underlying cause of schizophrenia. According to this hypothesis, any agent that can potentiate NMDA receptor currents, either directly by action on modulatory sites on the NMDA receptor (e.g., the glycine co-agonist binding site) or indirectly by activation of GPCRs known to potentiate NMDA receptor function (e.g. the M₁ mAChR), has the potential to ameliorate the symptoms of schizophrenia. In both preclinical and in clinical studies, Xanomeline, an M₁/M₄ preferring orthosteric agonist has proved efficacious with regard to positive, negative and cognitive symptoms, indicating that M₁ activation is a reasonable approach to the treatment of schizophrenia. More recently, the selective M₁ allosteric agonist TBPB demonstrated efficacy in multiple preclinical models of schizophrenia.

Cholinergic neurotransmission involves the activation of nictonic acetylcholine receptors (nAChRs) or the muscarinic acetylcholine receptors (mAChRs) by the binding of the endogenous orthosteric agonist acetylcholine (ACh). Clinical data supports that cholinergic hypofunction contributes to the cognitive deficits of patients suffering from AD and schizophrenia. As a result, acetylcholinesterase inhibitors, which inhibit the hydrolysis of ACh, have been approved in the United States for use in the palliative, but not disease-modifying, treatment of the cognitive deficits in AD patients. An alternative approach to pharmacologically target cholinergic hypofunction is the activation of mAChRs. mAChRs are widely expressed throughout the body. The mAChRs are members of the family A GPCRs and include five subtypes, designated M₁-M₅. M₁, M₃ and M₅ mainly couple to G_{q} and activate phospholipase C whereas M₂ and M₄ mainly couple to G_{i/o} and associated effector systems. These five distinct mAChR subtypes have been identified in the mammalian central nervous system where they are prevalent and differentially expressed. M₁-M₅ have varying roles in cognitive, sensory, motor and autonomic functions. Thus, without wishing to be bound by theory, it is believed that selective agonists of mAChR subtypes that regulate processes involved in cognitive function could prove superior to AChE inhibitors for treatment of AD and related disorders. The muscarinic M₁ receptor has been shown to have a major role in cognitive processing and is believed to have a major role in the pathophysiology of AD.

Evidence suggests that the most prominent adverse effects of AChE inhibitors and other cholinergic agents are mediated by activation of peripheral M₂ and M₃ mAChRs and include bradycardia, GI distress, excessive salivation, and sweating. In contrast, M₁ has been viewed as the most likely subtype for mediating the effects on cognition, attention mechanisms, and sensory processing. Because of this, considerable effort has been focused on developing selective M₁ agonists for treatment of AD. Unfortunately, these efforts have been largely unsuccessful because of an inability to develop compounds that are highly selective for the M₁ mAChR. Because of this, mAChR agonists that have been tested in clinical studies induce the same adverse effects of AChE inhibitors by activation of peripheral mAChRs. To fully understand the physiological roles of individual mAChR subtypes and to further explore the therapeutic utility of mAChR ligands in AD and other disorders, it can be important to develop compounds that are highly selective activators of M₁ and other individual mAChR subtypes.

Previous attempts to develop agonists that are highly selective for individual mAChR subtypes have failed because of the high conservation of the orthosteric ACh binding site. To circumvent problems associated with targeting the highly conserved orthosteric ACh site, a number of groups have shifted their focus to developing compounds that act at allosteric sites on mAChRs that are removed from the orthosteric site and are less highly-conserved. This approach is proving to be highly successful in developing selective ligands for multiple GPCR subtypes. In the case of mAChRs, a major goal has been to develop allosteric ligands that selectively increase activity of M₁ or other mAChR subtypes. Allosteric activators can include allosteric agonists, that act at a site removed from the orthosteric site to directly activate the receptor in the absence of ACh as well as positive allosteric modulators (PAMs), which do not activate the receptor directly but potentiate activation of the receptor by the endogenous othosteric agonist ACh. Also, it is possible for a single molecule to have both allosteric potentiator and allosteric agonist activity.

Phase III trials have shown that orthosteric mAChR activators can have efficacy in improving cognitive performance in AD patients. Moreover, data indicate that administration of M₁ activators decreases behavioral disturbances, including delusions, hallucinations, outbursts, and other symptoms in patients suffering from neurodegenerative diseases such as Alzheimer's disease. However, dose limiting adverse effects that may be due to lack of M1 mAChR selectivity led to failed launches of previous M1 agonists. In some cases, evidence suggests that mAChR activation also has the potential to be disease-modifying in that these agents may lower Aβ in AD patients. Interestingly, the M₁-selective allosteric agonist TBPB was found to display effects on the processing of APP toward the non-amyloidogenic pathway and decrease Aβ 1-40 and 1-42 production in vitro. These data suggest that selective activation of M₁ may provide a novel approach for both symptomatic and disease modifying treatment of Alzheimer's disease.

Despite advances in muscarinic receptor (mAChR) research, there is still a scarcity of compounds that are potent, efficacious, and selective activators of the M₁ mAChR that are also effective in the treatment of neurological and psychiatric disorders associated with cholinergic activity and diseases in which the muscarinic M₁ receptor is involved. These needs and other needs are satisfied by the present invention.

### SUMMARY

In accordance with the purpose(s) of the invention, as embodied and broadly described herein, the invention, in one aspect, relates to compounds useful as selective agonists of the M₁ receptor, which elicit receptor activation by binding at an allosteric site on the M₁ receptor, methods of making same, pharmaceutical compositions comprising same, and methods of treating disorders where selective M₁ activation would have a therapeutic benefit.

In one aspect, the invention relates to amidobipiperidinecarboxylate M1 allosteric agonists, analogs and derivatives thereof, and methods of making and using same (e.g., a class of alkyl 3-amido-1,4-biperidine-1-carboxylate compounds and their salts, pharmaceutical compositions comprising them and their use in therapy of the human body).

In a further aspect, the invention relates to a class of compounds that are muscarinic M₁ receptor allosteric agonists and therefore are useful in the treatment of Alzheimer's disease, schizophrenia, sleep disorders and other diseases in which selective activation of the muscarinic M₁ receptor would provide a therapeutic benefit.

Disclosed are compounds having a structure represented by a formula:

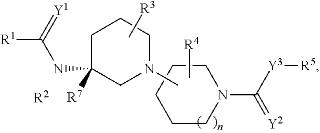

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

Also disclosed are methods for preparing a compound comprising the steps of providing an amino compound having a structure represented by a formula:

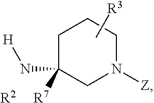

wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁷ is hydrogen or an optionally substituted organic residue comprising from 1 to 6 carbons; and Z is hydrogen or a protecting group, and reacting the amino compound with a carboxyl compound having a structure represented by a formula:

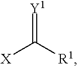

wherein Y¹ is O or S; wherein R¹ is an optionally substituted organic residue comprising from 1 to 12 carbons; and wherein X is a leaving group.

Also disclosed are methods for preparing a compound comprising the steps of: providing an amino compound having a structure represented by a formula:

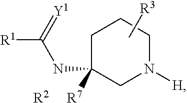

wherein Y¹ is O or S; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁷ is hydrogen or an optionally substituted organic residue comprising from 1 to 6 carbons, and reacting the amino compound under reductive amination conditions with a cycloalkanone compound having a structure represented by a formula:

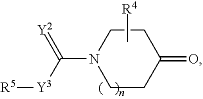

wherein n is an integer from 0 to 2; wherein Y² is O or S; wherein Y³ is a covalent bond, O, S, or N—R⁶; wherein R⁴ comprises from six to ten substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons.

Also disclosed are the products of the disclosed methods.

Also disclosed are pharmaceutical compositions comprising the product of a disclosed method and a pharmaceutically acceptable carrier.

Also disclosed are pharmaceutical compositions comprising a disclosed compound and a pharmaceutically acceptable carrier.

Also disclosed are methods for activating M₁ receptor activity in at least one cell comprising the step of contacting the at least one cell with at least one disclosed compound or at least one product of a disclosed method in an amount effective to activate M₁ receptor activity in the at least one cell.

Also disclosed are methods for activating M₁ receptor activity in a subject comprising the step of administering to the subject at least one disclosed compound or at least one product of a disclosed method in a dosage and amount effective to activating M₁ receptor activity in the subject.

Also disclosed are methods for the treatment of a disorder associated with cholinergic activity in a mammal comprising the step of administering to the mammal at least one disclosed compound or at least one product of a disclosed method in a dosage and amount effective to treat the disorder in the mammal.

Also disclosed are uses of a compound for M₁ receptor activation, the compound having a structure represented by a formula:

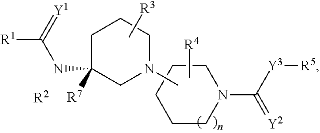

wherein n is an integer from 0 to 2; wherein Y¹ and Y² are independently O or S; wherein Y³ is a covalent bond, O, S, or N—R⁶⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

Also disclosed are kits comprising at least one compound having a structure represented by a formula:

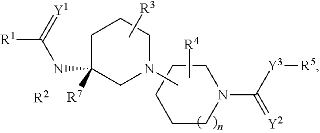

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, and one or more of:

a. at least one agent known to increase M₁ receptor activity;

b. at least one agent known to decrease M₁ receptor activity;

c. at least one agent known to treat a disorder associated with cholinergic activity;

d. instructions for treating a disorder associated with cholinergic activity;

e. instructions for treating a disorder associated with M₁ receptor activity; or

f. instructions for administering the compound in connection with cognitive or behavioral therapy.

In one aspect, the agent is known to have M₁ receptor agonist activity. In a further aspect, the agent is known to decrease M₁ receptor activity.

Also disclosed are methods for the manufacture of a medicament to activate the M₁ receptor in a mammal comprising combining at least one disclosed compound and/or product with a pharmaceutically acceptable carrier.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several embodiments and together with the description serve to explain the principles of the invention.

FIG. 1 is a graph showing the selectivity for M1 (>50 μM versus M2, M3, M4 and M5), by virtue of receptor activation at an allosteric site, as percent maximum acetylcholine response as a function of compound concentration for (R)-ethyl 3-(2-methylbenzamido)-1,4′-bipiperidine-1′-carboxylate (VU0364572).

FIG. 2 is a graph showing the selectivity for M1 (>50 μM versus M2, M3, M4 and M5), by virtue of receptor activation at an allosteric site, as percent maximum acetylcholine response as a function of compound concentration for (R)-ethyl 3-benzamido-1,4′-bipiperidine-1′-carboxylate (VU0359985).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where said event or circumstance occurs and instances where it does not.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by selective activation of the M₁ receptor” means having been subjected to a physical examination by a person of skill, for example, a physician, and found to have a condition that can be diagnosed or treated by a compound or composition that can favorably activate the M₁ receptor. As a further example, “diagnosed with a need for selective activatation of the M₁ receptor” refers to having been subjected to a physical examination by a person of skill, for example, a physician, and found to have a condition characterized by a deficit of M₁ receptor function. Such a diagnosis can be in reference to a disorder, such as a neurological and/or psychiatric disorder, obesity, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to M₁ receptor activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors - well known in the medical arts. For example, it is well within the skill of the art to start dose's of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of from 1 to 24 carbon atoms, for example from 1 to 12 carbons, from 1 to 8 carbons, from 1 to 6 carbons, or from 1 to 4 carbons, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including optionally substituted alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an interger from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

The term “organic residue” defines a carbon containing residue, i.e., a residue comprising at least one carbon atom, and includes but is not limited to the carbon-containing groups, residues, or radicals defined herein above. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

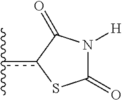

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “hydrolysable residue” is meant to refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (ee). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

In some aspects, a structure of a compound can be represented by a formula:

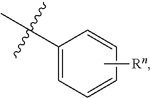

which is understood to be equivalent to a formula:

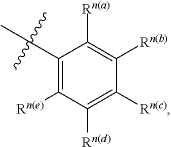

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds, or pharmaceutically acceptable derivatives thereof, useful as selective activators of the M₁ receptor, in this case allosteric agonists. In general, it is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, the invention relates to a compound having a structure represented by a formula:

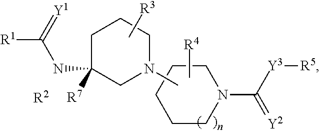

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

In a further aspect, the invention relates to a compound having a structure represented by a formula:

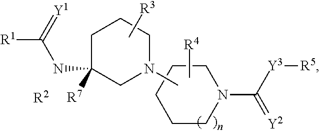

wherein n is an integer from 0 to 2; wherein Y¹ and Y² are independently O or S; wherein Y³ is O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons; and wherein R² is hydrogen or an optionally substituted organic residue comprising from 1 to 6 carbons, or a pharmaceutically acceptable derivative thereof.

In a further aspect, n is 1; wherein Y¹═Y²═Y³═O; and the compound has a structure represented by a formula:

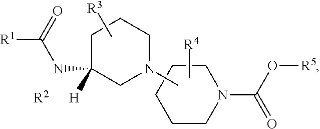

wherein R³ comprises eight substituents independently selected from hydrogen, halogen, hydroxyl, nitrile, nitro, thiol, optionally substituted amino, and optionally substituted organic residue comprising from 1 to 6 carbons, and wherein R⁴ comprises nine substituents independently selected from hydrogen, halogen, hydroxyl, nitrile, nitro, thiol, optionally substituted amino, and optionally substituted organic residue comprising from 1 to 6 carbons.

In a further aspect, n is 1; Y¹═Y²═Y³═O; all of R³ and R⁴ are hydrogen; and the compound has a structure represented by a formula:

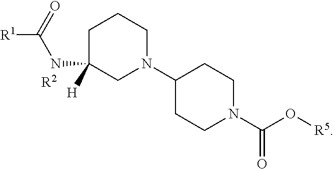

In a further aspect, n is 1; Y¹═Y²═Y³═O; all of R², R³, R⁴, and R⁷ are hydrogen; and the compound has a structure represented by a formula:

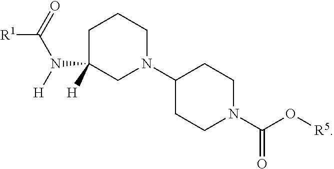

In a further aspect, n is 1; Y¹═Y²═Y³═O; all of R², R³, R⁴, and R⁷ are hydrogen; and the compound has a structure represented by a formula:

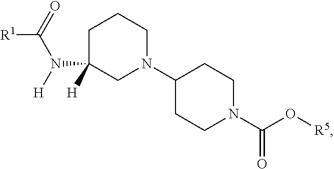

wherein R¹ is optionally substituted cycloalkyl, optionally substituted heterocycloalkyl, optionally substituted aryl, or optionally substituted heteroaryl; wherein R⁵ is selected from optionally substituted C1-C6 alkyl; and wherein the compound activates M₁ receptor response in M₁-transfected CHO-K1 cells, having an EC₅₀ of less than about 10 μM.

a. Y¹ and Y² Groups

In one aspect, Y¹ and Y² are independently O or S. That is, Y¹ can be O, or Y¹ can be S. Likewise, That is, Y² can be O, or Y² can be S. In certain aspects, Y¹═Y². In further aspects, Y¹═Y²═O, or Y¹═Y²═S. In a yet further aspect, Y¹═O, and Y²═S. In a still further aspect, Y¹═S, and Y²═O.

b. Y³ Groups

In one aspect, Y³ is a covalent bond, O, S, or N—R⁶. That is, Y³ can be O, Y³ can be N—R⁶, Y³ can be S, or Y³ can be a covalent bond. In a further aspect, Y³ is O, S, or N—R⁶. In certain aspects, Y¹═Y²═Y³═O. In a yet further aspect, Y¹═Y²═O, and Y³ is a colvant bond.

c. R¹ Groups

In one aspect, R¹ is an optionally substituted organic residue comprising 1 to 12 carbon atoms. In a further aspect, R¹ is selected from optionally substituted C1-C12 alkyl or C2-C12 alkenyl or C2-C12 alkynyl, optionally substituted C1-C12 heteroalkyl or C2-C12 heteroalkenyl or C2-C12 heteroalkynyl, optionally substituted C3-C12 cycloalkyl or C3-C12 cycloalkenyl, optionally substituted C3-C12 heterocycloalkyl or C3-C12 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl.

In a further aspect, R¹ is selected from 2-chlorobenzene, 2-methylbenzene, 3,4-difluorobenzenem, 3-fluorobenzene, 3-methoxybenzene, 4-fluoro-2-methylbenzene, 4-methoxybenzene, 4-methylbenzene, cyclohexane, cyclopentane, phenyl, and thiophene.

d. R² Groups

In one aspect, R² is hydrogen, a hydrolysable residue, or an optionally substituted organic residue comprising 1 to 6 carbons. In a further aspect, R² is selected from hydrogen, methyl, ethyl, propyl, butyl, pentyl, and hexyl. In a further aspect, R² is hydrogen or a hydrolysable residue. A hydrolysable residue can be a protecting group, for example, a butyloxycarbonyl group, including tBOC or nBOC.

e. R³ Groups

In one aspect, R³ comprises eight substituents independently selected from hydrogen, halogen, hydroxyl, nitrile, nitro, thiol, optionally substituted amino, and optionally substituted organic residue comprising from 1 to 6 carbons. In a further aspect, each R³ is selected from optionally substituted C1-C6 alkyl or C2-C6 alkenyl or C2-C6 alkynyl, optionally substituted C1-C6 heteroalkyl or C2-C6 heteroalkenyl or C2-C6 heteroalkynyl, optionally substituted C3-C6 cycloalkyl or C3-C6 cycloalkenyl, optionally substituted C3-C6 heterocycloalkyl or C3-C6 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, atkoxycarbonyl, carboxamide, amino-carbonyl, and alkylamine-carbonyl. In a further aspect, all of R³ are hydrogen.

f. R⁴ Groups

In one aspect, R⁴ comprises from seven to eleven substituents independently selected from hydrogen, halogen, hydroxyl, nitrile, nitro, thiol, optionally substituted amino, and optionally substituted organic residue comprising from 1 to 6 carbons. In a further aspect, n is 0, and R⁴ comprises seven substituents. In a further aspect, n is 1, and R⁴ comprises nine substituents. In a further aspect, n is 2, and R⁴ comprises eleven substituents.

In a further aspect, each R⁴ is selected from optionally substituted C1-C6 alkyl or C2-C6 alkenyl or C2-C6 alkynyl, optionally substituted C1-C6 heteroalkyl or C2-C6 heteroalkenyl or C2-C6 heteroalkynyl, optionally substituted C3-C6 cycloalkyl or C3-C6 cycloalkenyl, optionally substituted C3-C6 heterocycloalkyl or C3-C6 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl. In a further aspect, all of R⁴ are hydrogen.

In a further aspect, n is 1, and R⁴ comprises nine substituents independently selected from optionally substituted C1-C6 alkyl or C2-C6 alkenyl or C2-C6 alkynyl, optionally substituted C1-C6 heteroalkyl or C2-C6 heteroalkenyl or C2-C6 heteroalkynyl, optionally substituted C3-C6 cycloalkyl or C3-C6 cycloalkenyl, optionally substituted C3-C6 heterocycloalkyl or C3-C6 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl.

g. R⁵ Groups

In one aspect, R⁵ is hydrogen or an optionally substituted organic residue comprising from 1 to 12 carbons, with the proviso that wherein Y³ is a covalent bond, then R⁵ is hydrogen or optionally substituted C1-C6 alkyl. In a further aspect, R⁵ is an optionally substituted organic residue comprising 1 to 12 carbon atoms. In a further aspect, R⁵ is selected from optionally substituted C1-C12 alkyl or C2-C12 alkenyl or C2-C12 alkynyl, optionally substituted C1-C12 heteroalkyl or C2-C12 heteroalkenyl or C2-C12 heteroalkynyl, optionally substituted C3-C12 cycloalkyl or C3-C12 cycloalkenyl, optionally substituted C3-C12 heterocycloalkyl or C3-C12 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl. In a further aspect, R⁵ is selected from optionally substituted C1-C6 alkyl. In a further aspect, R⁵ is selected from methyl, ethyl, propyl, butyl, pentyl, hexyl, heptyl, octyl, nonyl, decyl, undecyl, or dodecyl. In a further aspect, R⁵ is selected from methyl, ethyl, propyl, butyl, pentyl, and hexyl. In a further aspect, R⁵ is ethyl. In a further aspect, R⁵ is hydrogen.

In a further aspect, R⁵ is hydrogen, with the proviso that Y³ is a covalent bond. In a further aspect, R⁵ is optionally substituted C1-C6 alkyl, with the proviso that Y³ is a covalent bond.

h. R⁶ Groups

In one aspect, R⁶, when present, is independently selected from hydrogen, a hydrolysable residue, and optionally substituted organic residue comprising from 1 to 6 carbon atoms. In a further aspect, R⁶ is selected from hydrogen, methyl, ethyl, propyl, butyl, pentyl, and hexyl. In a further aspect, R⁶ is hydrogen or a hydrolysable residue. A hydrolysable residue can be a protecting group, for example, a butyloxycarbonyl group, including tBOC or nBOC. In a further aspect, R⁶ is hydrogen.

i. R⁷ Groups

In one aspect, R⁷ is hydrogen or an optionally substituted organic residue comprising from 1 to 6 carbons. In a further aspect, R⁷ is hydrogen. In a further aspect, R⁷ is an optionally substituted organic residue comprising from 1 to 6 carbons. In a further aspect, R⁷ is selected from methyl, ethyl, propyl, butyl, pentyl, and hexyl.

j. Example Compounds

In one aspect, a compound can be present as:

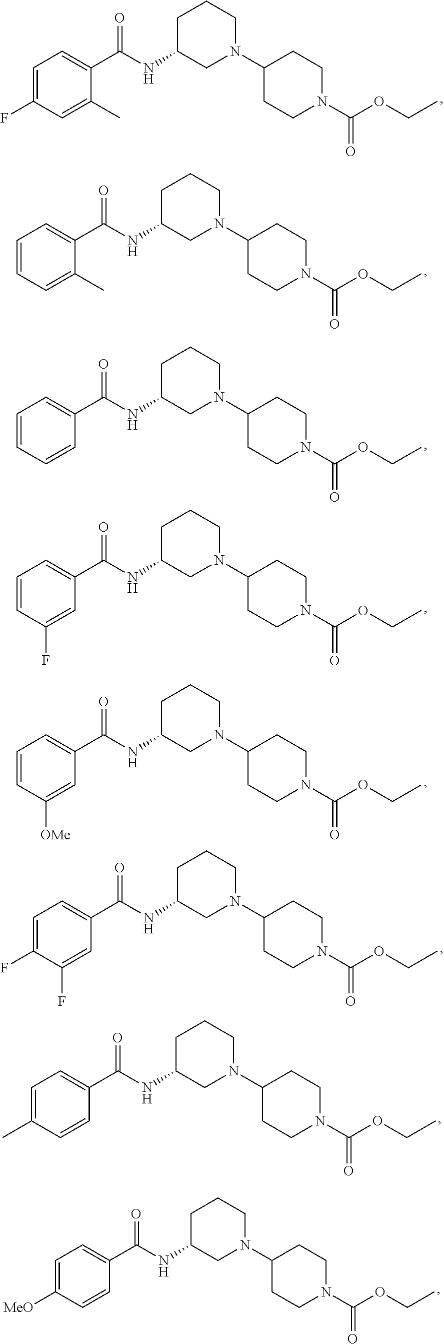

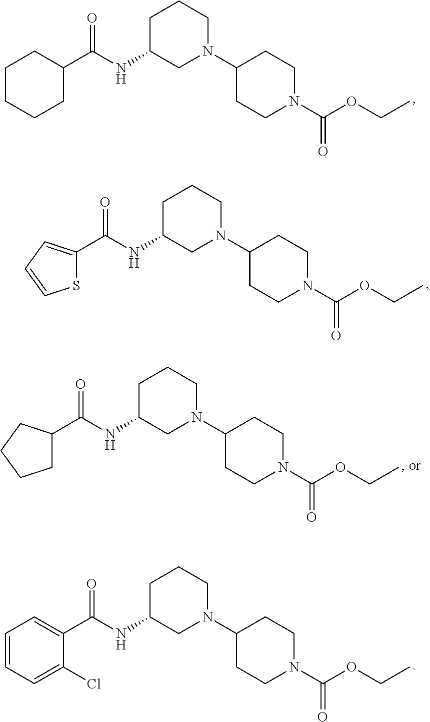

In one aspect, a compound can be present as present as:

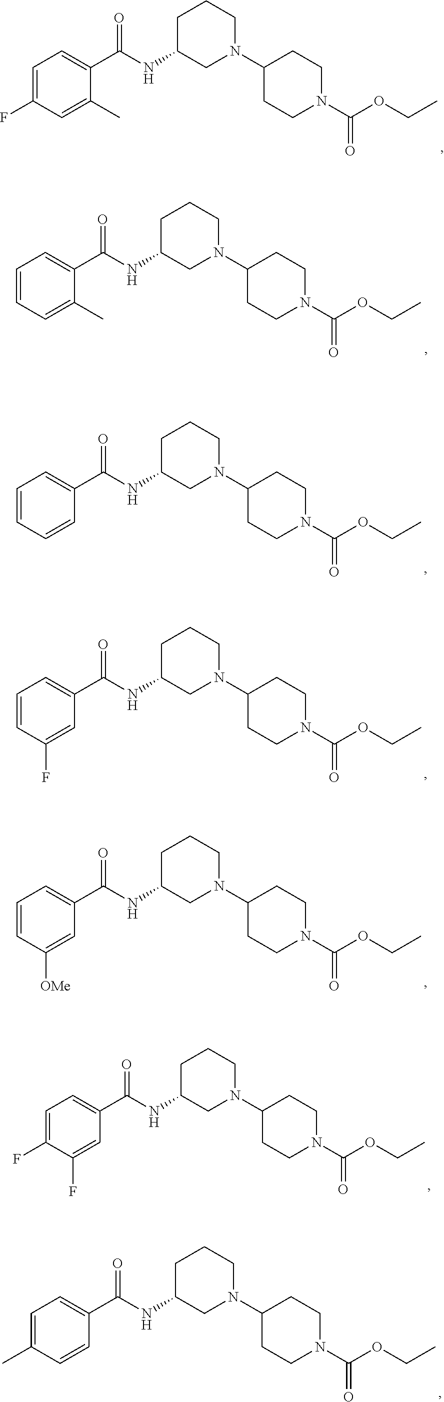

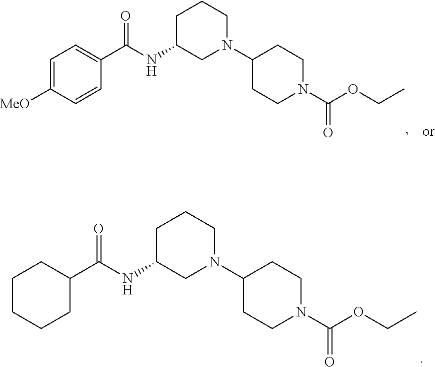

In one aspect, a compound can be present as:

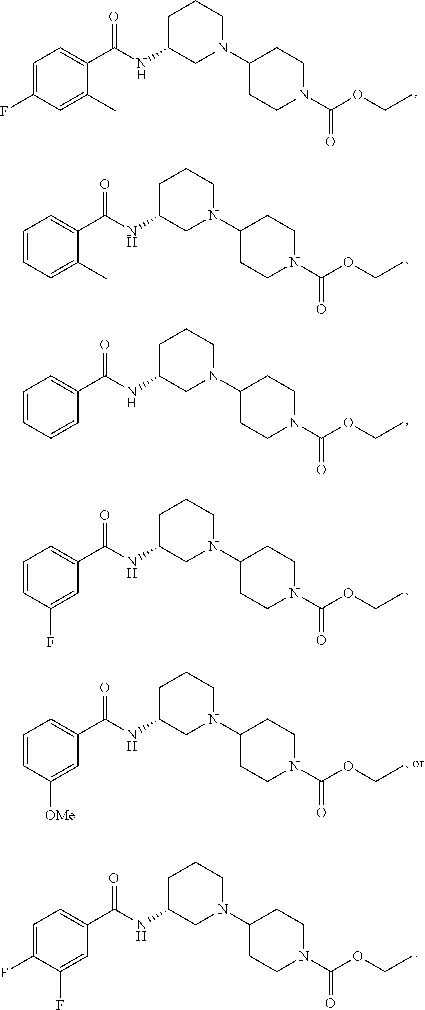

It is understood that the disclosed compounds can be used in connection with the disclosed methods, compositions, kits, and uses.

2. M₁ Activity

In one aspect, the compound activates M₁ response in M₁-transfected CHO-K1 cells. For example, the compound can have an EC₅₀ of less than about 10 μM, of less than about 5 μM, of less than about 1 μM, of less than about 500 nM, of less than about 100 nM, or of less than about 50 nM.

3. Enantiomer-Dependent Differential M1 Activity

In one aspect, the R-enantiomer of a disclosed compound activates M₁ response more potently than the corresponding S-enantiomer. For example, a particular R-enantiomer of a disclosed compound can have an EC₅₀ of less than about 10 μM, of less than about 5 μM, of less than about 1 μM, of less than about 500 nM, of less than about 100 nM, or of less than about 50 nM, while the corresponding S-enantiomer of the disclosed compound has an EC₅₀ of >10 μM.

While the disclosed compounds can be provided as a mixture of both the R-enantiomer and the S-enantiomer, it can be desired to provide the mixture of enantiomers of a disclosed compound enriched in the more potent compound. Such can be desired in order to, for example, increase the concentration of an active (or more active) enantiomer or in order to decrease the concentration of a less active (or inactive) enantiomer. Such can improve potency of a pharmaceutical preparation. Such also can minimize undesired side-effects present in a less active enantiomer and not present (or less present) in a more active enantiomer.

Thus, in various aspects, a disclosed compound can be provided in a form enriched in R-enantiomer of the compound. For example, a disclosed compound can be provided in an enantiomeric excess of greater than 50%, greater than 60%, greater than 70%, greater than 75%, greater than 80%, greater than 85%, greater than 90%, greater than 95%, greater than 98%, or greater than 99% of the R-enantiomer of the compound. In one aspect, the R-enantiomer is substantially free from the S-enantiomer. For example, the “R” forms of the compounds can be provided substantially free from the “S” forms of the compounds.

### C. Methods of Making the Compounds

In one aspect, the invention relates to methods of making compounds useful as selective allosteric activators of the M₁ receptor, which can be useful in the treatment disorder associated with M₁ receptor activity.

The compounds of this invention can be prepared by employing reactions as shown in the disclosed schemes, in addition to other standard manipulations that are known in the literature, exemplified in the experimental sections or clear to one skilled in the art. For clarity, examples having a fewer substituent can be shown where multiple substituents are allowed under the definitions disclosed herein.

An exemplary synthetic route is shown in Scheme 1 below:

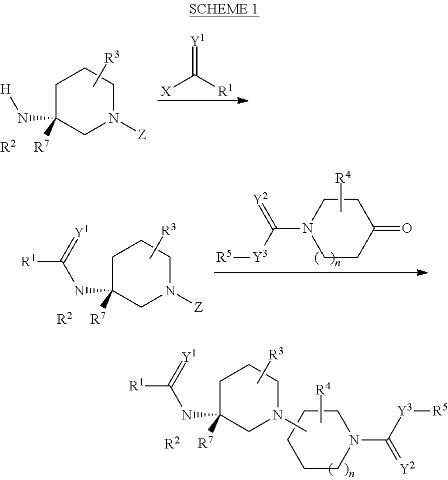

Generally, the disclosed methods can comprise one or both of two chemical transformations. The first transformation involves a reaction between an amine functionality and an activated carboxyl functionality, as represented in Scheme 2 below:

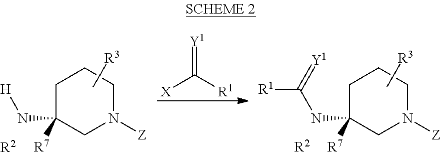

Both the starting amino compound and the activated carboxyl compound can be prepared or obtained commercially. A stereocenter is present in the amino compound. A particular steroisomer can be obtained during preparation of the amino compound by, for example, asymetric hydrogenation. If desired, a particular stereochemistry can be inverted from the corresponding alcohol compound by, for example, Mitsunobu inversion with an amine nucleophile. A particular steroisomer can be obtained by, for example, chiral separation of a racemic mixture of stereoisomers.

The product of this reaction can, thus, be an amide, which can be isolated or carried into another chemical transformation in unisolated form. In one aspect, Z can be a protecting group, which can be removed subsequent to this reaction as well as before, or concurrently with, further reaction.

The second transformation involves a reaction between a deprotected amino compound and a carbonyl compound under reductive amination conditions, as shown in Scheme 3:

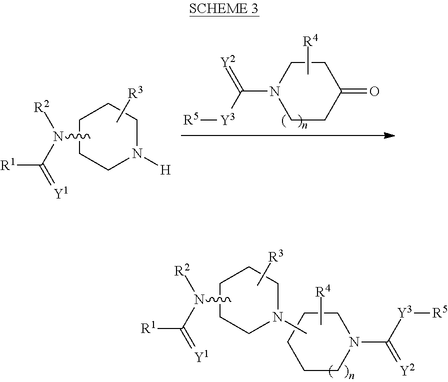

Thus, in one aspect, the invention relates to a method for preparing a compound comprising the steps of providing an amino compound having a structure represented by a formula:

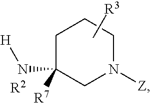

wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁷ is hydrogen or an optionally substituted organic residue comprising from 1 to 6 carbons; and Z is hydrogen or a protecting group, and reacting the amino compound with a carboxyl compound having a structure represented by a formula:

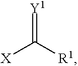

wherein Y¹ is O or S; wherein R¹ is an optionally substituted organic residue comprising from 1 to 12 carbons; and wherein X is a leaving group.

In a further aspect, providing comprises protecting the ring nitrogen to form the amino compound, wherein Z is a protecting group. In a further aspect, Z is a protecting group, wherein the protecting group is a butyloxycarbonyl group. In a further aspect, the leaving group is halide or pseudohalide.

In a further aspect, the amino compound has a structure represented by a formula:

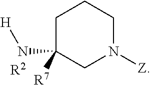

In a further aspect, the carboxyl compound has a structure represented by a formula:

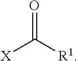

In one aspect, the method further comprises the step of reacting the product, wherein Z is hydrogen, under reductive amination conditions with a cycloalkanone compound having a structure represented by a formula:

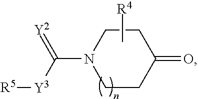

wherein n is an integer from 0 to 2; wherein Y² is O or S; wherein Y³ is a covalent bond, O, S, or N—R⁶; wherein R⁴ comprises from six to ten substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons, thereby forming a compound having a structure:

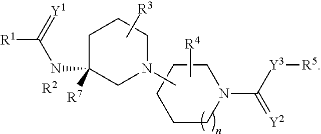

In a further aspect, the cycloalkanone compound has a structure represented by a formula:

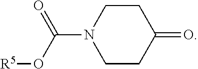

In a further aspect, the compound formed has a structure represented by a formula:

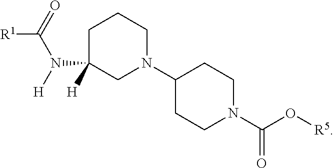

In one aspect, the invention relates to a method for preparing a compound comprising the steps of providing an amino compound having a structure represented by a formula:

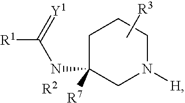

wherein Y¹ is O or S; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁷ is hydrogen or an optionally substituted organic residue comprising from 1 to 6 carbons, and reacting the amino compound under reductive amination conditions with a cycloalkanone compound having a structure represented by a formula:

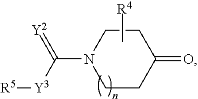

wherein n is an integer from 0 to 2; wherein Y² is O or S; wherein Y³ is O, S, or N—R⁶; wherein R⁴ comprises from six to ten substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons.

In a further aspect, the amino compound has a structure represented by a formula:

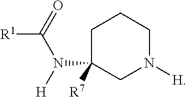

In a further aspect, the carboxyl compound has a structure represented by a formula:

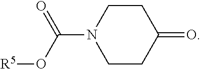

In a further aspect, providing comprises reacting an amino compound having a structure represented by a formula:

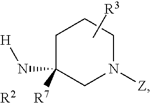

wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁷ is hydrogen or an optionally substituted organic residue comprising from 1 to 6 carbons; and Z is hydrogen or a protecting group, with a carboxyl compound having a structure represented by a formula:

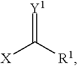

wherein Y¹ is O or S; wherein R¹ is an optionally substituted organic residue comprising from 1 to 12 carbons; and wherein X is a leaving group, and optionally, removing the protecting group Z, when present.

In a further aspect, Z is a protecting group, wherein the protecting group is a butyloxycarbonyl group, and wherein the optional removal step is performed.

In a further aspect, the compound formed has a structure represented by a formula:

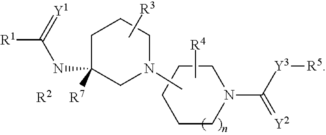

In a further aspect, the compound formed has a structure represented by a formula:

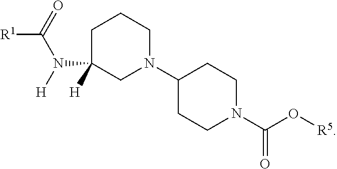

In one aspect, the resultant compound activates M₁ response as an increase in calcium fluorescence in M₁-transfected CHO-K1 cells in the presence of the compound, compared to the response of equivalent CHO-K1 cells in the absence of the compound, having an EC₅₀ of less than about 100 μM, for example, less than about 1 μM.

It is understood that the disclosed methods can be used in connection with the disclosed compounds, compositions, kits, and uses.

### D. Pharmaceutical Compositions

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable derivatives (e.g., salt(s)) thereof) as an active ingredient, a pharmaceutically acceptable carrier, and, optionally, other therapeutic ingredients or adjuvants. The compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The disclosed compounds can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and can be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans. The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions encompass any composition made by admixing a compound of the present invention and a pharmaceutically acceptable carrier.

As used herein, the term “pharmaceutically acceptable salts” refers to salts prepared from pharmaceutically acceptable non-toxic bases or acids. When a disclosed compound is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable derivatives thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment of conditions which require activation of M₁, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The disclosed pharmaceutical compositions can further comprise other therapeutically active compounds, as discussed further herein, which are usually applied in the treatment of the above mentioned pathological conditions.

It is understood that the disclosed compositions can be prepared from the disclosed compounds. It is also understood that the disclosed compositions can be employed in the disclosed methods of using.

### E. Kits

In one aspect, the invention relates to kits comprising at least one disclosed compound or at least one product of a disclosed method and at least one agent known to have M₁ receptor agonist activity. Also disclosed are kits comprising at least one compound having a structure represented by a formula:

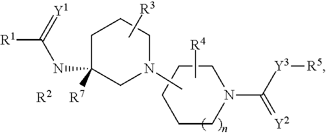

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, and one or more of: a. at least one agent known to increase M₁ receptor activity; b. at least one agent known to decrease M₁ receptor activity; c. at least one agent known to treat a disorder associated with cholinergic activity; d. instructions for treating a disorder associated with cholinergic activity; e. instructions for treating a disorder associated with M₁ receptor activity; or f. instructions for administering the compound in connection with cognitive or behavioral therapy.

In a further aspect, the invention relates to kits comprising at least one compound having a structure represented by a formula:

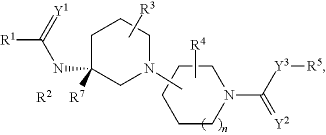

wherein n is an integer from 0 to 2; wherein Y¹ and Y² are independently O or S; wherein Y³ is O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile; nitro, thiol, optionally substituted amino, and optionally substituted organic residue comprising from 1 to, 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, and one or more of: a. at least one agent known to increase M₁ receptor activity; b. at least one agent known to decrease M₁ receptor activity; c. at least one agent known to treat a disorder associated with cholinergic activity; d. instructions for treating a disorder associated with cholinergic activity; e. instructions for treating a disorder associated with M₁ receptor activity; or f. instructions for administering the compound in connection with cognitive or behavioral therapy.

In one aspect, the agent is known to have M₁ receptor agonist activity. In a further aspect, the agent is known to decrease M₁ receptor activity. In a further aspect, the at least one compound or the at least one product and the at least one agent are co-formulated. In a further aspect, the at least one compound or the at least one product and the at least one agent are co-packaged.

### F. Methods of Using the Compounds and Compositions

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

In one aspect, the compounds can be coadministered with anti-Alzheimer's agents, beta-secretase inhibitors, gamma-secretase inhibitors, orthosteric muscarinic agonists, muscarinic potentiators, cholinesterase inhibitors, HMG-CoA reductase inhibitors, NSAIDs and anti-amyloid antibodies. In a further aspect, the compounds can be administered in combination with sedatives, hypnotics, anxiolytics, antipsychotics (typical and atypical), selective serotonin reuptake inhibitors (SSRIs), monoamine oxidase inhibitors (MAOIs), 5-HT2 antagonists, GlyT1 inhibitors and the like such as, but not limited to: risperidone, clozapine, haloperidol, fluoxetine, prazepam, xanomeline, lithium, phenobarbitol, and salts thereof and combinations thereof.

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating, preventing, ameliorating, controlling or reducing the risk of a variety of disorders associated with selective M₁ receptor activation. For example, a treatment can include selective M₁ receptor activation to an extent effective to affect cholinergic activity. Thus, a disorder can be associated with cholinergic activity, for example cholinergic hypofunction. Thus, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders associated with M₁ receptor activity in a subject comprising the step of administering to the subject at least one disclosed compound; at least one disclosed pharmaceutical composition; and/or at least one disclosed product in a dosage and amount effective to treat the disorder in the subject.

For example, in one aspect, the invention relates to a method for treating a disorder associated with selective M1 receptor activation, for example, a disorder associated with cholinergic activity, in a mammal comprising the step of administering to the mammal at least one compound having a structure represented by a formula:

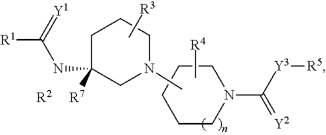

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, in a dosage and amount effective to treat the disorder in the mammal.

In a further aspect, the invention relates to a method for the treatment of a disorder associated with selective M1 receptor activation, for example, a disorder associated with cholinergic activity, in a mammal comprising the step of administering to the mammal at least one compound having a structure represented by a formula:

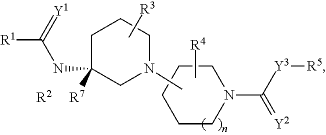

wherein n is an integer from 0 to 2; wherein Y¹ and Y² are independently O or S; wherein Y³ is O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, in a dosage and amount effective to treat the disorder in the mammal.

In one aspect, the method treats a disorder associated with selective M₁ receptor activation. In a further aspect, the method treats a disorder associated with cholinergic activity. In a further aspect, the method treats a disorder associated with cholinergic activity by selective M₁ receptor activation. In a further aspect, the method selectively activates M₁ receptor, thereby treating a disorder associated with cholinergic activity.

In a yet further aspect, the invention relates to a method for the treatment of a disorder associated with cholinergic activity, in a mammal comprising the step of administering to the mammal at least one compound having a structure represented by a formula:

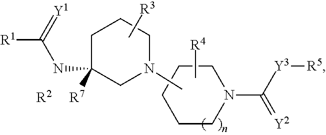

wherein n is an integer from 0 to 2; wherein Y¹ and Y² are independently O or S; wherein Y³ is O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, in a dosage and amount effective to selectively activate M1 receptor, thereby treating the disorder in the mammal.

The invention is directed at the use of described chemical compositions to treat diseases or disorders in patients (preferably human) wherein the M₁ receptor is involved, such as Alzhiemer's disease (both palliative cognitive and disease-modifying), cognitive impairment, schizophrenia, pain disorders (including acute pain, neuropathic pain and inflammatory pain), and sleep disorders, by administering one or more disclosed compounds or products.

In one aspect, provided is a method for treating or preventing anxiety, comprising: administering to a subject at least one disclosed compound; at least one disclosed pharmaceutical composition; and/or at least one disclosed product in a dosage and amount effective to treat the disorder in the subject. At present, the fourth edition of the Diagnostic and Statistical Manual of Mental Disorders (DSM-IV) (1994, American Psychiatric Association, Washington, D.C.), provides a diagnostic tool for disorders including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified.

Also provided is a method for the treatment of a disorder in a mammal comprising the step of administering to the mammal at least one disclosed compound, composition, or medicament.

In one aspect, the NMDA receptor is central to a wide range of CNS processes, and plays a role in a variety of disease states in humans or other species. The action of the M₁ receptor potentiates NMDA receptor function, which increases activation of the NMDA receptor following glutamate release from the presynaptic terminal. Changes in NMDA-mediated neurotransmission have been implicated in certain neuropsychiatric disorders such as dementia, depression and psychoses, for example schizophrenia, and learning and memory disorders, for example attention deficit disorders and autism.

In one aspect, the disclosed compounds have utility in treating a variety of neurological and psychiatric disorders associated with the M₁ receptor, including one or more of the following conditions or diseases: schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anaesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age-related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; NMDA receptor-related disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de Ia Tourette's syndrome, epilepsy, muscular spasms and disorders associated with muscular spasticity or weakness including tremors; dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxysmal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

In a specific aspect, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources. In another specific embodiment, the present invention provides a method for treating anxiety disorders, comprising: administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In a further specific aspect, the present invention provides a method for treating schizophrenia or psychosis comprising: administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-W-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

In a further specific aspect, the present invention provides a method for treating substance-related disorders and addictive behaviors, comprising: administering to a patient in need thereof aneffective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In a still further aspect, the present invention provides a method for treating pain, comprising: administering to a patient in need thereof an effective amount of a compound of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

In a further aspect, the present invention provides a method for treating obesity or eating disorders associated with excessive food intake and complications associated therewith, comprising: administering to a patient in need thereof an effective amount of a compound of the present invention. At present, obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington DC) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-W-TR. The skilled artisan will recognize that there are alternative nomenclatures, nosologies and classification systems for general medical conditions, and that these systems evolve with medical and scientific progress. Thus, the term “obesity or eating disorders associated with excessive food intake” is intended to include like conditions and disorders that are described in other diagnostic sources.

The compounds are further useful in a method for the prevention, treatment, control, amelioration, or reducation of risk of the diseases, disorders and conditions noted herein. The compounds are further useful in a method for the prevention, treatment, control, amelioration, or reduction of risk of the aforementioned diseases, disorders and conditions in combination with other agents.

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present invention.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1;1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In one aspect, the compound can be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies. In another embodiment, the subject compound can be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazeparn, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, Zolpidem, and salts thereof, and combinations thereof, and the like, or the subject compound can be administered in conjunction with the use of physical methods such as with light therapy or electrical stimulation.

In a further aspect, the compound can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In a further aspect, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In one aspect, the compound can be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HTJA agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In the treatment of conditions which require activation of M1 an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in one aspect, the invention relates to a method for activating M1 receptor activity in at least one cell comprising the step of contacting the at least one cell with at least one disclosed compound or at least one product of a disclosed method in an amount effective to activate M₁ receptor in the at least one cell. In a further aspect, the cell is mammalian, for example, human. In a further aspect, the cell has been isolated from a subject prior to the contacting step. In a further aspect, contacting is via administration to a subject.

In a further aspect, the invention relates to a method for activating M1 receptor activity in a subject comprising the step of administering to the subject at least one disclosed compound or at least one product of a disclosed method in a dosage and amount effective to activating M1 receptor activity in the subject. In a further aspect, the subject is mammalian, for example, human. In a further aspect, the mammal has been diagnosed with a need for M₁ receptor agonism prior to the administering step. In a further aspect, the mammal has been diagnosed with a need for M₁ receptor activation prior to the administering step. In a further aspect, the method further comprises the step of identifying a subject in need of M₁ receptor agonism.

In a further aspect, the invention relates to a method for the treatment of a disorder associated with selective M1 receptor activation, for example, a disorder associated with cholinergic activity, in a mammal comprising the step of administering to the mammal at least one disclosed compound or at least one product of a disclosed method in a dosage and amount effective to treat the disorder in the mammal. In a further aspect, the mammal is a human. In a further aspect, the mammal has been diagnosed with a need for treatment for the disorder prior to the administering step. In a further aspect, the method further comprises the step of identifying a subject in need of treatment for the disorder.

In one aspect, the disorder can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, conduct disorder, autistic disorder; movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders. In a further aspect, the disorder is Alzheimer's disease. In a further aspect, the disorder is a neurological and/or psychiatric disorder associated with M₁ receptor activity dysfunction.

It is understood that the disclosed methods can be used in connection with the disclosed compounds, compositions, kits, and uses.

2. Cotherapeutic Methods

The present invention is further directed to administration of a selective M₁ receptor activator for improving treatment outcomes in the context of cognitive or behavioral therapy. That is, in one aspect, the invention relates to a cotherapeutic method comprising the step of administering to a mammal an effective amount and dosage of at least one compound having a structure represented by a formula:

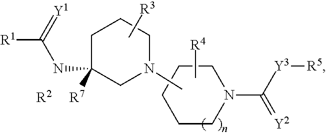

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof, in connection with cognitive or behavioral therapy.

In a further aspect, adminstration improves treatment outcomes in the context of cognitive or behavioral therapy. Adminstration in connection with cognitive or behavioral therapy can be continuous or intermittent. Adminstration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9,or 10 half-lives of the administered compound.

It is understood that the disclosed cotherapeutic methods can be used in connection with the disclosed compounds, compositions, kits, and uses.

3. Manufacture of a Medicament

The present invention is further directed to a method for the manufacture of a medicament for activating M₁ receptor (e.g., treatment of one or more neurological and/or psychiatric disorder associated with M₁ dysfunction) in mammals (e.g., humans) comprising combining one or more disclosed compounds, products, or compositions with a pharmaceutically acceptable carrier or diluent.

For example, in one aspect, a medicament can comprise one or more compounds having a structure represented by a formula:

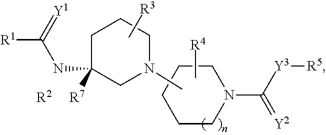

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

In a further aspect, a medicament can comprise one or more compounds having a structure represented by a formula:

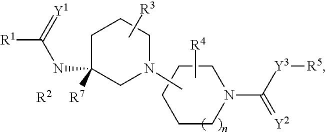

wherein n is an integer from 0 to 2; wherein Y¹ and Y² are independently O or S; wherein Y³ is O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

4. Use of Compounds

In one aspect, the invention relates to the use of a compound for M₁ receptor activation, the compound having a structure represented by a formula:

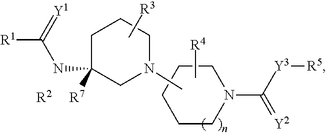

wherein n is an integer from 0 to 2; wherein Y¹ and Y² are independently O or S; wherein Y³ is a covalent bond, O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

In a further aspect, the invention relates to the use of a compound for M₁ receptor activation, the compound having a structure represented by a formula:

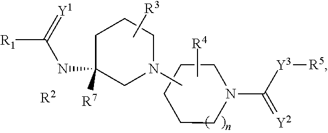

wherein n is an integer from 0 to 2; wherein Y¹ and Y² are independently O or S; wherein Y³ is O, S, or N—R⁶; wherein R¹ is an optionally substituted organic residue comprising from 1 to 12 carbons; wherein R² is hydrogen, a hydrolysable residue, or an optionally substituted organic residue comprising 1 to 6 carbons; wherein R³ comprises eight substituents independently selected from hydrogen, halogen, hydroxyl, nitrile, nitro, thiol, optionally substituted amino, and optionally substituted organic residue comprising from 1 to 6 carbons; wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons; wherein R⁵ is an optionally substituted organic residue comprising from 1 to 12 carbons; wherein R⁶, when present, is independently selected from hydrogen, a hydrolysable residue, and optionally substituted organic residue comprising from 1 to 6 carbons; and wherein R⁷ is hydrogen or an optionally substituted organic residue comprising from 1 to 6 carbons, or a pharmaceutically acceptable derivative thereof.

In a further aspect, n is 1; Y¹═Y²═Y³═O; and the compound has a structure represented by a formula:

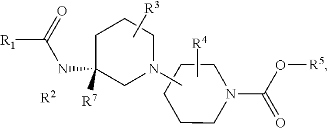

wherein R³ comprises eight substituents independently selected from hydrogen, halogen, hydroxyl, nitrile, nitro, thiol, optionally substituted amino, and optionally substituted organic residue comprising from 1 to 6 carbons, and wherein R⁴ comprises nine substituents independently selected from hydrogen, halogen, hydroxyl, nitrile, nitro, thiol, optionally substituted amino, and optionally substituted organic residue comprising from 1 to 6 carbons.

In a further aspect, n is 1; Y¹═Y²═Y³═O; all of R³, R⁴, and R⁷ are hydrogen; and the compound has a structure represented by a formula:

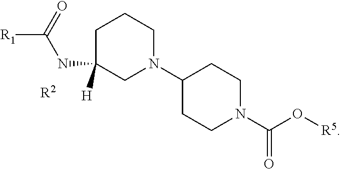

In a further aspect, the compound is used for the treatment of a disorder associated with cholinergic activity in a mammal. In a further aspect, the compound activates M₁ receptor response activity, having an EC₅₀ of less than about 10 μM.

In a further aspect, the compound activates M₁ receptor in M₁-transfected CHO-K1 cells, having an EC₅₀ of less than about 10 μM. In a further aspect, the compound is used for the treatment of a disorder associated with cholinergic activity in a mammal. In one aspect, the use relates to a treatment of a disorder in a mammal. In one aspect, the use is characterized in that the mammal is a human. In one aspect, the use is characterized in that the disorder is a disorder associated with muscarinic type 1 receptor (M₁) activity.

It is understood that the disclosed uses can be employed in connection with the disclosed compounds, methods, compositions, and kits.

5. Allosteric Activation of the M₁ Receptor

Also provided is a method for allosteric activation (allosteric agonism) of the M₁ recepotor in at least one cell comprising the step of contacting the at least one cell with at least one disclosed compound in an amount effective to activate M₁ receptor activity in the at least one cell. In a further aspect, provided is a method for activation (allosteric agonism) of the M₁ receptor in a subject, the method comprising the step of administering to the subject a therapeutically effective amount of at least one disclosed compound, in a dosage and amount effective to activate M₁ receptor activity in the subject. In a further aspect, the method can be applied to a subject, e.g., a mammal, including, for example, a human.

6. Subjects

The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a disorder treatable by activation of the M₁ receptor and/or a need for activation of M₁ receptor activity prior to the administering step. In some aspects of the disclosed method, the subject has been diagnosed with anxiety or a related disorder prior to the administering step. In some aspects of the disclosed methods, the subject has been identified with a need for treatment prior to the administering step. In some aspects of the disclosed method, the subject has been identified with a disorder treatable by activation of the M1 receptor and/or or a need for activation/agonism of M₁ activity prior to the administering step. In some aspects of the disclosed method, the subject has been identified with anxiety or a related disorder prior to the administering step. In one aspect, a subject can be treated prophylactically with a compound or composition disclosed herein, as discussed herein elsewhere.

### G. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following examples. Starting materials and the requisite intermediates are in some cases commercially available, or can be prepared according to literature procedures or as illustrated herein. All ¹H NMR spectra were obtained on instrumentation at a field strength of 300 to 500 MHz.

### 1. SYNTHESES OF ALKYL 3-AMIDO-1,4-BIPIPERIDINE-1-CARBOXYLATES

Compounds were prepared according to the following procedures:

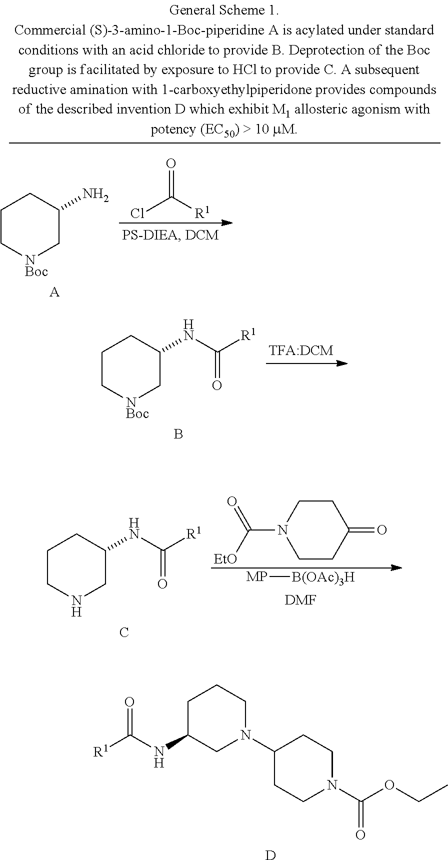

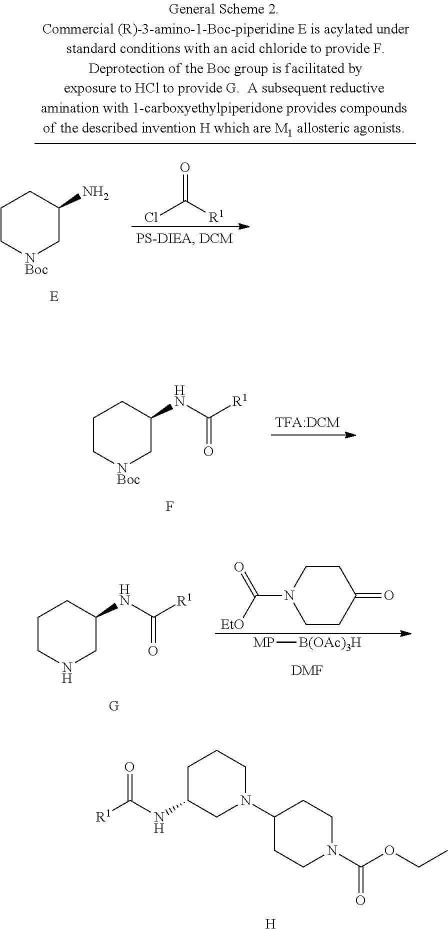

### a. SYNTHESIS OF I-4: (S)-ETHYL 3-(2-METHYLBENZAMIDO)-1,4′-BIPEIPERIDINE-1′-CARBOXYLATE

To a solution of o-toluoyl chloride (37.6 mg, 0.243 mmol) in dichloromethane (2 mL) was added (S)-(+)-3-amino-1-boc-piperidine (38.5 mg, 0.221 mmol) and PS-DIEA (2 eq, 126.3 mg). The reaction was agitated overnight at room temperature, after which the reaction mixture was filtered to remove polymer-supported resin. The filtrate was then concentrated and dissolved in 1 mL of dichloromethane, to which was added 1 mL of trifluoroacetic acid (1:1 DCM:TFA) and stirred at room temperature for 30 min. Following deprotection, the reaction mixture was again concentrated to dryness, after which the product was dissolved in dimethylformamide (2 mL). To this solution was added 1-carbethoxy-4-piperidone (41.7 mg, 0.243 mmol) and MP-BH(OAc)₃ (2.5 eq, 229 mg). The reaction mixture was then agitated overnight at room temperature, after which filtration and sample concentration yielded (S)-ethyl 3-(2-methylbenzamido)-1,4′-bipiperidine-1′-carboxylate as a clear oil. The product was then dissolved in 1 mL of DMSO and purified by mass-directed preparative HPLC to afford (S)-ethyl 3-(2-methylbenzamido)-1,4′-bipiperidine-1′-carboxylate 2,2,2-trifluoroacetate as a clear oil (10.3 mg, 12.5%); ¹H-NMR (400 MHz, d₆-DMSO) δ 10.6 (br s, 1H), 8.38 (d, J=7.60 Hz, 1H), 7.36-7.21 (m, 5H), 4.11 (br d, J=12.0 Hz, 2H), 4.04 (q, J=6.8 Hz, 2H), 3.51-3.37 (m, 6H), 2.71 (q, J=10.4 Hz, 1H), 2.34 (s, 3H), 2.09 (t, J=12.4 Hz, 4H), 1.63-1.50 (m, 4H), 1.18 (t, J=6.8 Hz, 3H); m/z 374.2 [M+H].

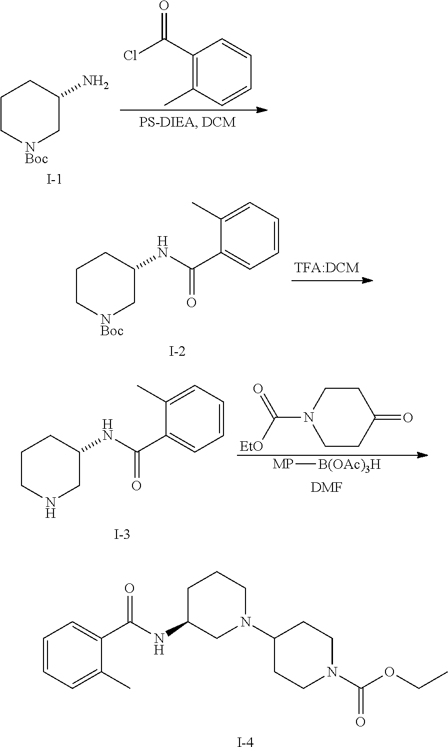

### b. SYNTHESIS OF II-4: (R)-ETHYL 3-(2-METHYLBENZAMIDO)-1,4′-BIPEIPERIDINE-1′-CARBOXYLATE

To a solution of o-toluoyl chloride (37.6 mg, 0.243 mmol) in dichloromethane (2 mL) was added (R)-(+)-3-amino-1-boc-piperidine (38.5 mg, 0.221 mmol) and PS-DIEA (2 eq, 126.3 mg). The reaction was agitated overnight at room temperature, after which the reaction mixture was filtered to remove polymer-supported resin. The filtrate was then concentrated and dissolved in 1 mL of dichloromethane, to which was added 1 mL of trifluoroacetic acid (1:1 DCM:TFA) and stirred at room temperature for 30 min. Following deprotection, the reaction mixture was again concentrated to dryness, after which the product was dissolved in dimethylformamide (2 mL). To this solution was added 1-carbethoxy-4-piperidone (41.7 mg, 0.243 mmol) and MP-BH(OAc)₃ (2.5 eq, 229 mg). The reaction mixture was then agitated overnight at room temperature, after which filtration and sample concentration yielded (R)-ethyl 3-(2-methylbenzamido)-1,4′-bipiperidine-1′-carboxylate as a clear oil. The product was then dissolved in 1 mL of DMSO and purified by mass-directed preparative HPLC to afford (R)-ethyl 3-(2-methylbenzamido)-1,4′-bipiperidine-1′-carboxylate 2,2,2-trifluoroacetate as a clear oil (10.3 mg, 12.5%); ¹H-NMR (400 MHz, d₆-DMSO) δ 10.6 (br s, 1H), 8.38 (d, J=7.60 Hz, 1H), 7.36-7.21 (m, 5H), 4.11 (br d, J=12.0 Hz, 2H), 4.04 (q, J=6.8 Hz, 2H), 3.51-3.37 (m, 6H), 2.71 (q, J=10.4 Hz, 1H), 2.34 (s, 3H), 2.09 (t, J=12.4 Hz, 4H), 1.63-1.50 (m, 4H), 1.18 (t, J=6.8 Hz, 3H); m/z 374.2 [M+H].

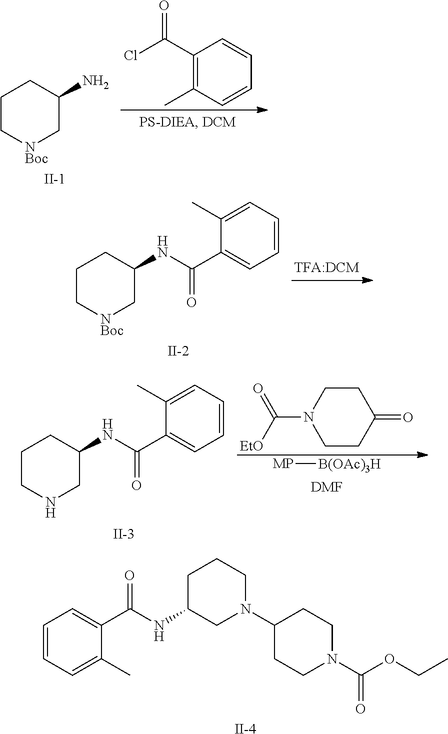

The following examples were each synthesized according to General Scheme 2.

### c. SYNTHESIS OF III-3: (R)—N-(PIPERIDIN-3-YL)THIOPHENE-2-CARBOXAMIDE

To a solution of 2-thiopheneacetyl chloride (110.0 mg, 0.75 mmol) in dichloromethane (2 mL) was added (R)-(+)-3-amino-1-boc-piperidine (100.0 mg, 0.50 mmol) and PS-DIEA (2 eq, 207 mg). The reaction was agitated overnight at room temperature, after which the reaction mixture was filtered to remove polymer-supported resin. The filtrate was then scavenged for excess acid chloride for 45 min at room temperature with stirring using PS-Trisamine (3.5 eq relative to excess acid chloride). The mixture was then filtered, concentrated to dryness and dissolved in 1 mL of dichloromethane, to which was added 1 mL of trifluoroacetic acid (1:1 DCM:TFA) and stirred at room temperature for 30 min to affect deprotection. Following deprotection, the reaction mixture was again concentrated to dryness to yield (R)—N-(piperidin-3-yl)thiophene-2-carboxamide as a viscous oil (74 mg, 70.4%); ¹H-NMR (400 MHz, d₆-DMSO) δ 8.11 (d, J=8.0, 1H), 7.77 (m, 1H), 7.71 (m, 1H), 7.11 (m, 1H), 3.71 (m, 1H) 2.93 (m, 1H), 2.77 (m, 1H), 2.36 (m, 2H), 1.84 (m, 1H), 1.60 (m, 1H), 1.4 (m, 3H); m/z 211.2 [M+H].

### d. SYNTHESIS OF III-4: (R)-ETHYL 3-(THIOPHENE-2-CARBOXAMIDO)-1,4′-BIPIPERIDINE-1′-CARBOXYLATE

To a solution of (R)—N-(piperidin-3-yl)thiophene-2-carboxamide (54 mg, 0.257 mmol) in dimethylformamide (1.5 mL) was added 1-carbethoxy-4-piperidone (40 mg, 0.234 mmol) and MP-BH(OAc)₃ (2.5 eq, 250 mg). The reaction mixture was then agitated overnight at room temperature, filtered, and concentrated. The crude product was then re-dissolved in CHCl₂ (3 mL) to which PS-Isocyanate (3.0 equivalents relative to excess amine) was added, and the solution was stirred for 1 hour and then filtered. The solution was next loaded onto an SCX cartridge, washed with MeOH (3×10 mL), and then eluted with 2M NH₃ in MeOH (2×5 mL) after which sample concentration yielded (R)-ethyl 3-(thiophene-2-carboxamido)-1,4′-bipiperidine-1′-carboxylate as a clear viscous oil (27 mg, 32%); ¹H-NMR (400 MHz, d₆-DMSO) δ 8.13 (br d, J=7.60 Hz, 1H), 7.76 (d, J=3.6 Hz, 1H), 7.71 (d, J=5.0 Hz, 1H), 7.11 (t, J=3.8 Hz, 1H), 4.07 (q, J=13.5, 5.2 Hz, 1H), 3.99 (m, 3H), 3.90 (m, 1H), 3.15 (d, J=5.2 Hz, 2H), 2.86 (m, 1H), 2.69 (m, 3H), 2.08 (m, 2H), 1.78 (m, 1H), 1.67 (m, 3H), 1.43 (m, 1H), 1.30 (m, 2H), 1.15 (t, J=7.1 Hz, 3H); m/z 366.2 [M+H].

### e. SYNTHESIS OF IV-3: (R)—N-(PIPERIDIN-3-YL)CYCLOHEXANECARBOXAMIDE

To a solution of cyclohexanecarbonyl chloride (187.4 mg, 1.28 mmol) in dichloromethane (2 mL) was added (R)-(+)-3-amino-1-boc-piperidine (256.04 mg, 1.28 mmol) and PS-DIEA (2 eq, 730 mg). The reaction was agitated overnight at room temperature, after which the reaction mixture was filtered to remove polymer-supported resin. The filtrate was then scavenged for excess acid chloride for 45 min at room temperature with stirring using PS-Trisamine (3.5 eq relative to excess acid chloride). The mixture was then filtered, concentrated to dryness and dissolved in 1 mL of dichloromethane, to which was added 1 mL of trifluoroacetic acid (1:1 DCM:TFA) and stirred at room temperature for 30 min to affect deprotection. Following deprotection, the reaction mixture was again concentrated to dryness to yield (R)—N-(piperidin-3-yl)cyclohexanecarboxamide as a viscous oil (78 mg, 74.2%); (400 MHz, d₆-DMSO) δ 7.42 (br d, J=7.8, 1H), 3.48 (m, 1H), 3.16 (s, 1H), 2.81 (m, 1H), 2.70 (m, 1H), 2.36 (m, 1H), 2.20 (m, 1H), 2.05 (m, 1H), 1.61 (m, 7H), 1.22 (m, 7H); m/z 211.2 [M+H].

### f. SYNTHESIS OF IV-4: (R)-ETHYL 3-(CYCLOHEXANECARBOXAMIDO)-1,4′-BIPIPERIDINE-1′-CARBOXYLATE

To a solution of (R)—N-(piperidin-3-yl)cyclohexanecarboxamide (54 mg, 0.257 mmol) in dimethylformamide (1.5 mL) was added 1-carbethoxy-4-piperidone (40 mg, 0.234 mmol) and MP-BH(OAc)₃ (2.5 eq, 250 mg). The reaction mixture was then agitated overnight at room temperature, filtered, and concentrated. The crude product was then re-dissolved in CHCl₂ (3 mL) to which PS-Isocyanate (3.0 equivalents relative to excess amine) was added, and the solution was stirred for 1 hour and then filtered. The solution was next loaded onto an SCX cartridge, washed with MeOH (3×10 mL), and eluted with 2M NH₃ in MeOH (2×5 mL) after which sample concentration yielded (R)-ethyl 3-(cyclohexanecarboxamido)-1,4′-bipiperidine-1′-carboxylate as a clear viscous oil (22 mg, 26%); ¹H-NMR (400 MHz, d₆-DMSO) δ 7.41 (br d, J=6.0 Hz, 1H), 4.0 (m, 4H), 3.6 (m, 1H), 2.65 (m, 4H), 2.40 (m, 1H), 2.00 (m, 2H), 1.90 (m, 1H), 1.60 (m, 9H), 1.30 (m, 9H), 1.15 (t, J=7.0 Hz, 3H); m/z 366.3 [M+H].

### g. SYNTHESIS OF V-3: (R)-3-METHOXY-N-(PIPERIDIN-3-YL)BENZAMIDE

To a solution of 3-methoxybenzoyl chloride (100 mg, 0.586 mmol) in dichloromethane (2 mL) was added (R)-(+)-3-amino-1-boc-piperidine (117.4 mg, 0.586 mmol) and PS-DIEA (2 eq, 335 mg). The reaction was agitated overnight at room temperature, after which the reaction mixture was filtered to remove polymer-supported resin. The filtrate was then scavenged for excess acid chloride for 45 min at room temperature with stirring using PS-Trisamine (3.5 eq relative to excess acid chloride). The mixture was then filtered, concentrated to dryness and dissolved in 1 mL of dichloromethane, to which was added 1 mL of trifluoroacetic acid (1:1 DCM:TFA) and stirred at room temperature for 30 min to affect deprotection. Following deprotection, the reaction mixture was again concentrated to dryness to yield (R)-3-methoxy-N-(piperidin-3-yl)benzamide as a viscous oil (65 mg, 55.5%); ¹H-NMR (400 MHz, d₆-DMSO) δ 8.07 (br d, J=7.9 Hz, 1H), 7.37 (m, 3H), 7.06 (m, 1H), 3.79 (m, 4H), 3.16 (s, 1H), 2.94 (m, 1H), 2.77 (m, 1H), 2.37 (m, 2H), 1.81 (m, 1H), 1.59 (m, 1H), 1.43 (m, 2H); m/z 235.2 [M+H].

### h. SYNTHESIS OF V-4: (R)-ETHYL 3-(3-METHOXYBENZAMIDO)-1,4′-BIPIPERIDINE-1′-CARBOXYLATE

To a solution of (R)-3-methoxy-N-(piperidin-3-yl)benzamide (60 mg, 0.257 mmol) in dimethylformamide (1.5 mL) was added 1-carbethoxy-4-piperidone (40 mg, 0.234 mmol) and MP-BH(OAc)₃ (2.5 eq, 250 mg). The reaction mixture was then agitated overnight at room temperature, filtered, and concentrated. The crude product was then re-dissolved in CHCl₂ (3 mL) to which PS-Isocyanate (3.0 equivalents relative to excess amine) was added, and the solution was stirred for 1 hour and then filtered. The solution was next loaded onto an SCX cartridge, washed with MeOH (3×10 mL), and eluted with 2M NH₃ in MeOH (2×5 mL) after which sample concentration yielded (R)-ethyl 3-(3-methoxybenzamido)-1,4′-bipiperidine-1′-carboxylate as a clear viscous oil (31 mg, 34%); ¹H-NMR (400 MH^{z}, d₆-DMSO) δ 8.05 (br d, J=7.2 Hz, 1H), 7.35 (m, 3H), 7.06 (m, 1H), 4.07 (q, J=10.5, 5.2 Hz, 1H), 3.99 (m, 3H), 3.79 (s, 3H), 3.16 (d, J=5.2 Hz, 3H), 2.87 (br d, J=8.0 Hz, 1H), 2.72 (m, 3H), 2.10 (m, 2H), 1.76 (m, 1H), 1.68 (m, 3H), 1.45 (m, 3H), 1.35 (t, J=7.1, 3H); m/z 390.3 [M+H].

### i. SYNTHESIS OF VI-3: (R)-3,4-DIFLUORO-N-(PIPERIDIN-3-YL)BENZAMIDE

To a solution of 3,4-difluorobezoyl chloride (100 mg, 0.566 mmol) in dichloromethane (2 mL) was added (R)-(+)-3-amino-1-boc-piperidine (113.4 mg, 0.566 mmol) and PS-DIEA (2 eq, 323 mg). The reaction was agitated overnight at room temperature, after which the reaction mixture was filtered to remove polymer-supported resin. The filtrate was then scavenged for excess acid chloride for 45 min at room temperature with stirring using PS-Trisamine (3.5 eq relative to excess acid chloride). The mixture was then filtered, concentrated to dryness and dissolved in 1 mL of dichloromethane, to which was added 1 mL of trifluoroacetic acid (1:1 DCM:TFA) and stirred at room temperature for 30 min to affect deprotection. Following deprotection, the reaction mixture was again concentrated to dryness to yield (R)-3,4-difluoro-N-(piperidin-3-yl)benzamide as a viscous oil (77 mg, 64.1%); ¹H-NMR (400 MHz, d₆-DMSO) δ 8.20 (br d, J=7.8 Hz, 1H), 7.88 (m, 1H), 7.73 (m, 1H), 7.52 (m, 1H), 3.76 (m, 1H), 3.16 (s, 1H), 2.94 (m, 1H), 2.74 (d, J=12.4 Hz, 1H), 2.37 (m, 2H), 1.83 (m, 1H), 1.62 (m, 1H), 1.35 (m 2H); m/z 241.2 [M+H].

### j. SYNTHESIS OF VI-4: (R)-ETHYL 3-(3,4-DIFLUOROBEZAMIDO)-1,4′-BIPIPERIDINE-1′-CARBOXYLATE

To a solution of (R)-3,4-difluoro-N-(piperidin-3-yl)benzamide (62 mg, 0.257 mmol) in dimethylformamide (1.5 mL) was added 1-carbethoxy-4-piperidone (40 mg, 0.234 mmol) and MP-BH(OAc)₃ (2.5 eq, 250 mg). The reaction mixture was then agitated overnight at room temperature, filtered, and concentrated. The crude product was then re-dissolved in CHCl₂ (3 mL) to which PS-Isocyanate (3.0 equivalents relative to excess amine) was added, and the solution was stirred for 1 hour and then filtered. The solution was next loaded onto an SCX cartridge, washed with MeOH (3×10 mL), and eluted with 2M NH₃ in MeOH (2×5 mL) after which sample concentration yielded (R)-ethyl 3-(3,4-difluorobezamido)-1,4′-bipiperidine-1′-carboxylate as a clear viscous oil (31 mg, 33%); ¹H-NMR (400 MH^{z}, d₆-DMSO) δ 8.23 (br d, J=7.4 Hz, 1H), 7.88 (m, 1H), 7.72 (m, 1H) 7.53 (m, 1H), 4.07 (q, J=10.5, 5.2 Hz, 1H) 4.00 (m, 4H), 3.84 (m, 1H), 3.16 (d, J=5.2 Hz, 2H), 2.88 (m, 1H), 2.71 (m, 3H), 2.08 (m, 2H), 1.78 (m, 1H), 1.67 (m, 2H), 1.45 (m, 1H), 1.28 (m, 2H), 1.15 (t, J=7.1, 3H); m/z 396.3 [M+H].

### k. SYNTHESIS OF VII-3: (R)-2-METHYL-N-(PIPERIDIN-3-YL)BENZAMIDE

To a solution of o-toluoyl chloride (100 mg, 0.647 mmol) in dichloromethane (2 mL) was added (R)-(+)-3-amino-1-boc-piperidine (129.5 mg, 0.647 mmol) and PS-DIEA (2 eq, 370 mg). The reaction was agitated overnight at room temperature, after which the reaction mixture was filtered to remove polymer-supported resin. The filtrate was then scavenged for excess acid chloride for 45 min at room temperature with stirring using PS-Trisamine (3.5 eq relative to excess acid chloride). The mixture was then filtered, concentrated to dryness and dissolved in 1 mL of dichloromethane, to which was added 1 mL of trifluoroacetic acid (1:1 DCM:TFA) and stirred at room temperature for 30 min to affect deprotection. Following deprotection, the reaction mixture was again concentrated to dryness to yield (R)-2-methyl-N-(piperidin-3-yl)benzamide as a viscous oil (87 mg, 79.7%); ¹H-NMR (400 MHz, d₆-DMSO) δ 8.01 (br d, J=7.9 Hz, 1H), 7.24 (m, 4H), 3.73 (m, 1H), 3.16 (s, 1H), 2.95 (m, 1H), 2.76 (m, 1H), 2.36 (m, 2H), 2.30 (m, 3H), 1.82 (m, 1H), 1.60 (m, 1H), 1.39 (m, 2H); m/z 219.2 [M+H].

### l. SYNTHESIS OF VII-4: (R)-ETHYL 3-(2-METHYLBENZAMIDO)-1,4′-BIPIPERIDINE-1′-CARBOXYLATE

To a solution of (R)-2-methyl-N-(piperidin-3-yl)benzamide (56 mg, 0.257 mmol) in dimethylformamide (1.5 mL) was added 1-carbethoxy-4-piperidone (40 mg, 0.234 mmol) and MP-BH(OAc)₃ (2.5 eq, 250 mg). The reaction mixture was then agitated overnight at room temperature, filtered, and concentrated. The crude product was then re-dissolved in CHCl₂ (3 mL) to which PS-Isocyanate (3.0 equivalents relative to excess amine) was added, and the solution was stirred for 1 hour and then filtered. The solution was next loaded onto an SCX cartridge, washed with MeOH (3×10 mL), and eluted with 2M NH₃ in MeOH (2×5 mL) after which sample concentration yielded (R)-ethyl 3-(2-methylbenzamido)-1,4′-bipiperidine-1′-carboxylate as a clear viscous oil (24 mg, 27%); ¹H-NMR (400 MHz, d₆-DMSO) δ 8.00 (br d, J=10 Hz, 1H), 7.23 (m, 4H), 4.07 (q, J=10.5, 5.2 Hz, 1H), 4.00 (q, J=14.1, 7.1 Hz, 4H), 3.83 (m, 1H), 3.23 (d, J=5.2, 2H), 2.86 (m, 1H), 2.69 (m, 3H), 2.30 (s, 3H), 2.10 (m, 2H), 1.76 (m, 1H), 1.68 (m, 2H), 1.44 (m, 1H), 1.28 (m, 2H), 1.16 (t, J=7.1, 3H); m/z 374.3 [M+H].

### m. SYNTHESIS OF VIII-3: (R)—N-(PIPERIDIN-3-YL)BENZAMIDE

To a solution of benzoyl chloride (406 mg,, 0.711 mmol) in dichloromethane (2 mL) was added (R)-(+)-3-amino-1-boc-piperidine (142.5 mg, 0.711 mmol) and PS-DIEA (2 eq, 406 mg). The reaction was agitated overnight at room temperature, after which the reaction mixture was filtered to remove polymer-supported resin. The filtrate was then scavenged for excess acid chloride for 45 min at room temperature with stirring using PS-Trisamine (3.5 eq relative to excess acid chloride). The mixture was then filtered, concentrated to dryness and dissolved in 1 mL of dichloromethane, to which was added 1 mL of trifluoroacetic acid (1:1 DCM:TFA) and stirred at room temperature for 30 min to affect deprotection. Following deprotection, the reaction mixture was again concentrated to dryness to yield (R)—N-(piperidin-3-yl)benzamide as a viscous oil (70 mg, 68.5%); ¹H-NMR (400 MHz, d₆-DMSO) δ 8.09 (br d, J=7.8 Hz, 1H), 7.81 (m, 2H), 7.46 (m, 3H), 3.78 (m, 1H), 3.16 (s, 1H), 2.94 (m, 1H), 2.75 (m, 1H), 2.38 (m, 2H), 1.82 (m, 1H), 1.61 (m, 1H), 1.44 (m, 2H); m/z 205.2 [M+H].

### n. SYNTHESIS OF VIII-4: (R)-ETHYL 3-BENZAMIDO-1,4′-PIPERIDINE-1′-CARBOXYLATE

To a solution of (R)—N-(piperidin-3-yl)benzamide (53 mg, 0.257 mmol) in dimethylformamide (1.5 mL) was added 1-carbethoxy-4-piperidone (40 mg, 0.234 mmol) and MP-BH(OAc)₃ (2.5 eq, 250 mg). The reaction mixture was then agitated overnight at room temperature, filtered, and concentrated. The crude product was then re-dissolved in CHCl₂ (3 mL) to which PS-Isocyanate (3.0 equivalents relative to excess amine) was added, and the solution was stirred for 1 hour and then filtered. The solution was next loaded onto an SCX cartridge, washed with MeOH (3×10 mL), and eluted with 2M NH₃ in MeOH (2×5 mL) after which sample concentration yielded (R)-ethyl 3-benzamido-1,4′-bipiperidine-1′-carboxylate as a clear viscous oil (35 mg, 41%); ¹H-NMR (400 MHz, d₆-DMSO) δ8.10 (br s, 1H), 7.82 (d, J=7.1, 2H), 7.49 (m, 2H), 7.44 (m, 1H), 4.80 (q, J=10.5, 5.2 Hz, 1H), 4.02 (q, J=14.1, 7.1 Hz, 4H), 3.88 (m, 1H), 3.31 (s, 2H), 2.88 (m, 1H), 2.72 (m, 3H), 2.11 (br s, 2H), 1.71 (m, 3H), 1.35 (m, 3H), 1.15 (m, 3H) ; m/z 360.3 [M+H].

### 2. CELL CULTURE AND UPTAKE MEASUREMENTS

Cell culture and transfections. Chinese hamster ovary (CHO-K1) cells stably expressing rat (r)M₁ were purchased from the American Type Culture Collection and cultured according to their indicated protocol. CHO cells stably expressing human (h)M₂, hM₃, and hM₅ were used and described previously (Levey et al., 1991); rM₄ cDNA, provided by T. I. Bonner (National Institutes of Health, Bethesda, Md.), was used to stably transfect CHO-K1 cells purchased from the American Type Culture Collection using Lipofectamine2000. To make stable hM₂ and rM₄ cell lines for use in calcium mobilization assays, these cells also were stably transfected with a chimeric G-protein (Gqi5) using Lipofectamine 2000. M₁, hM₃, and hM₅ cells were grown in Ham's F-12 medium containing 10% heat-inactivated fetal bovine serum (FBS), 20 mM HEPES, and 50 μg/ml G418 sulfate. hM₂-Gqi5 cells were grown in the same medium also containing 500 μg/ml Hygromycin B. Stable rM₄-Gqi5 cells were grown in DMEM containing 10% heat-inactivated FBS, 20 mM HEPES, 400 μg/ml G418 sulfate, and 500 μg/ml Hygromycin B. The rat M₁ Y381A orthosteric mutant receptor cDNA was generated using the Quik-Change site-directed mutagenesis kit (Stratagene) and verified by sequencing. CHO-K1 cells were stably transfected with this cDNA using Lipofectamine2000 and screened for expression based on calcium mobilization in response to the allosteric M₁ agonist N-desmethylclozapine.

Calcium mobilization assays. For measurement of agonist-evoked increases in intracellular calcium, CHO-K1 cells stably expressing muscarinic receptors were plated in 100 μl of growth medium at 5×10⁴ (rM1, hM3, and hM5) or 6×10⁴ cells per well (rM1 Y381A, hM₂, and rM₄) in Costar 96-well black-walled, tissue culture (TC)-treated, clear-bottom plates (Fisher). Cells were incubated overnight at 37° C. and 5% CO₂. The next day, medium was removed from the cells, and they were incubated with 50 μl of 2 μM Fluo-4 AM diluted in assay buffer [HBSS (Invitrogen) supplemented with 20 mM HEPES and 2.5 mM probenecid, pH 7.4] for 1 h at 37° C. Dye was removed and replaced with 45 μl of assay buffer. Agonists were diluted into assay buffer at a 2× concentration and applied to cells using the automated system at 19 s into the 130 s protocol. Calcium flux was measured over time as an increase in fluorescence (fold over basal) using the Flexstation plate reader (Molecular Devices).

### 3. POTENCY RESULTS FOR EXEMPLARY ALKYL 3-AMIDO-1,4-BIPIPERIDINE-1-CARBOXYLATE M1 ALLOSTERIC AGONISTS

The potency of exemplary disclosed compounds was established using the above-described procedure. The results for R-enantiomer compounds are shown in Table 1.

TABLE 1
POTENCY RESULTS

| | | Efficacy | | |
|---|---|---|---|---|
| | Potency | (% Max | | |
| Structure | (EC₅₀) | ACh) | Name | (M + H) |
| 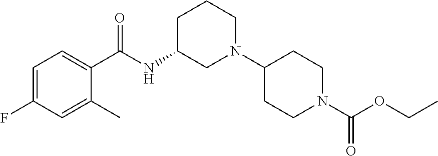 | 59 nM | 77 | (R)-ethyl 3-(4-fluoro- 2-methylbenzamido)- 1,4′-bipiperidine-1′- carboxylate | 392.2 |
| 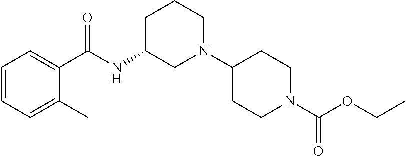 | 88 nM | 96 | (R)-ethyl 3-(2- methylbenzamido)- 1,4′-bipiperidine-1′- carboxylate | 374.2 |
| 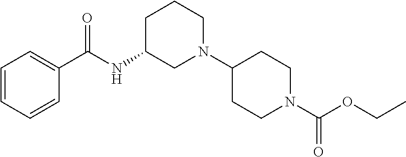 | 127 nM | 91 | (R)-ethyl 3- benzamido-1,4′- bipiperidine-1′- carboxylate | 360.3 |
| 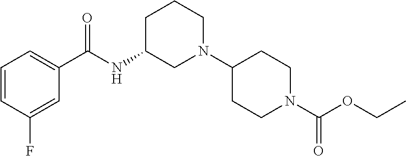 | 144 nM | 92 | (R)-ethyl 3-(3- fluorobenzamido)- 1,4′-bipiperidine-1′- carboxylate | 378.3 |
| 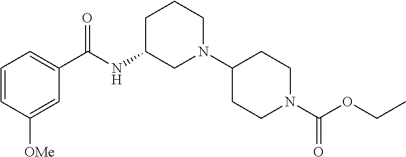 | 245 nM | 91 | (R)-ethyl 3-(3- methoxybenzamido)- 1,4′-bipiperidine-1′- carboxylate | 390.2 |
| 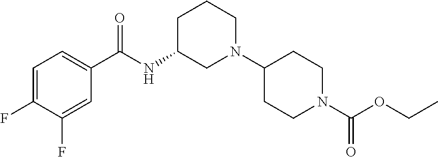 | 309 nM | 84 | (R)-ethyl 3-(3,4- difluorobenzamido)- 1,4′-bipiperidine-1′- carboxylate | 396.2 |
| 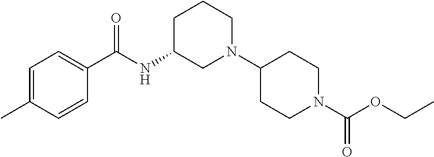 | 819 nM | 14 | (R)-ethyl 3-(4- methylbenzamido)- 1,4′-bipiperidine-1′- carboxylate | 374.3 |
| 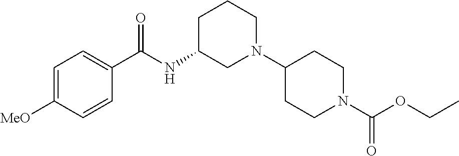 | 963 nM | 63 | (R)-ethyl 3-(4- methoxybenzamido)- 1,4′-bipiperidine-1′- carboxylate | 390.2 |
| 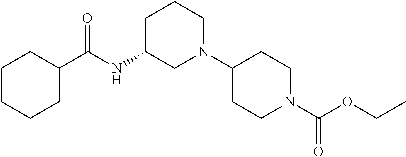 | 685 nM | 44 | (R)-ethyl 3- (cyclohexane- carboxamido)- 1,4′-bipiperidine- 1′-carboxylate | 366.3 |
| 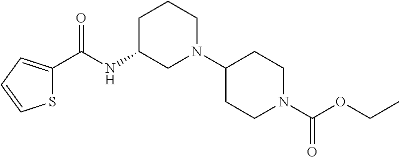 | 1.3 μM | 61 | (R)-ethyl 3-(thiophene- 2-carboxamido)-1,4′- bipiperidine-1′- carboxylate | 366.2 |
| 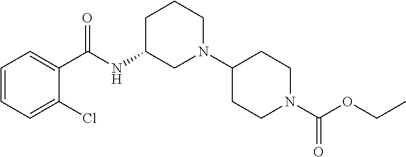 | 2.6 μM | 57 | (R)-ethyl 3-(2- chlorobenzamido)- 1,4′-bipiperidine-1′- carboxylate | 394.2 |
| 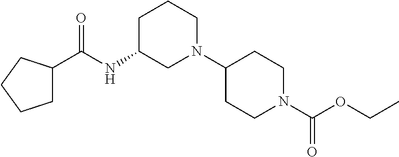 | 5.3 μM | 35 | (R)-ethyl 3- (cyclopentane- carboxamido)-1,4′- bipiperidine- 1′-carboxylate | 352.3 |

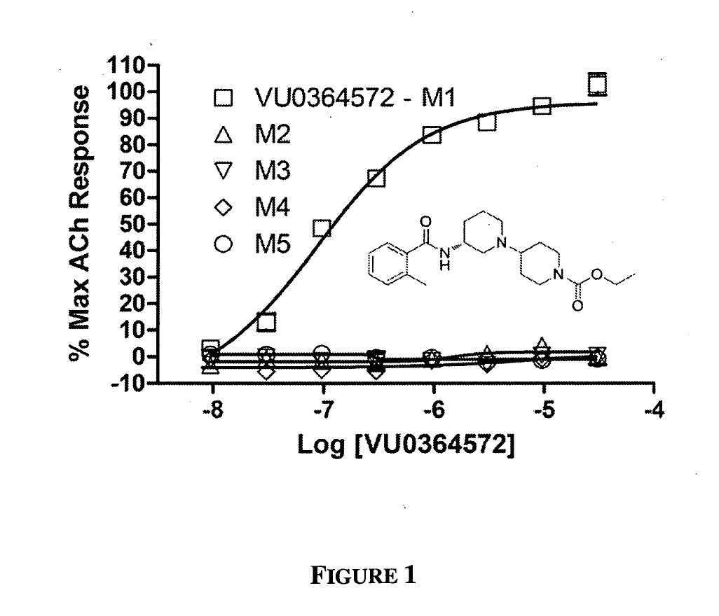

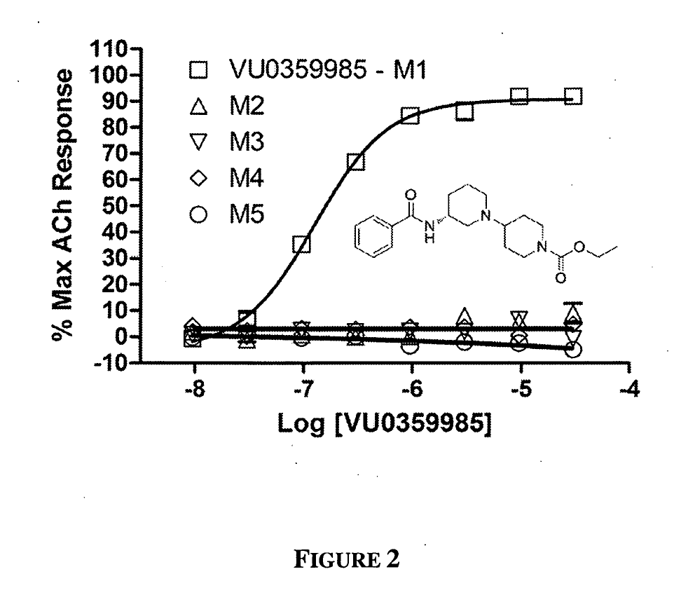

Compounds listed in Table 1 are selective for M1 (>50 μM potency (EC₅₀) versus M2, M3, M4 and M5) by virtue of receptor activation at an allosteric site. Examples of the mAChR selectivity are shown in FIG. 1 and FIG. 2.

The results for S-enantiomer compounds are shown in Table 2.

TABLE 2
POTENCY RESULTS

| | Potency | | |
|---|---|---|---|
| Structure | (EC₅₀) | Name | (M + H) |
| 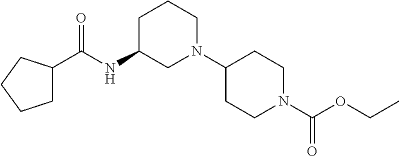 | >10 μM | (S)-ethyl 3- (cyclopentane- carboxamido)- 1,4′-bipiperidine-1′- carboxylate | 352.3 |
| 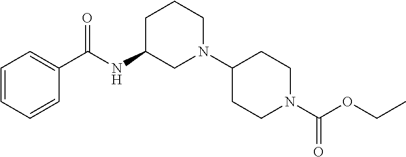 | >10 μM | (S)-ethyl 3-benzamido-1,4′- bipiperidine-1′-carboxylate | 360.3 |
| 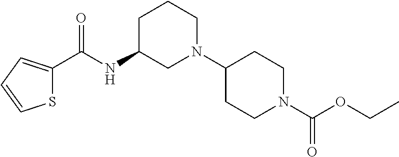 | >10 μM | (S)-ethyl 3-(thiophene-2- carboxamido)-1,4′- bipiperidine-1′-carboxylate | 366.2 |
| 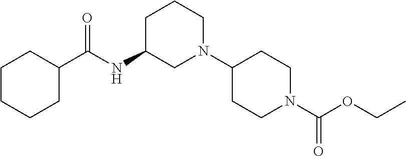 | >10 μM | (S)-ethyl 3- (cyclohexanecarboxamido)- 1,4′-bipiperidine-1′- carboxylate | 366.3 |
| 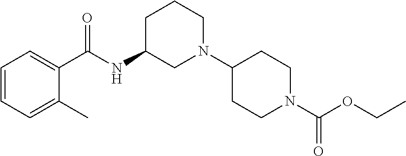 | >10 μM | (S)-ethyl 3-(2- methylbenzamido)-1,4′- bipiperidine-1′-carboxylate | 374.2 |
| 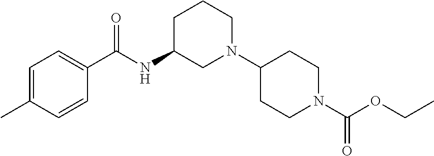 | >10 μM | (S)-ethyl 3-(4- methylbenzamido)-1,4′- bipiperidine-1′-carboxylate | 374.3 |
| 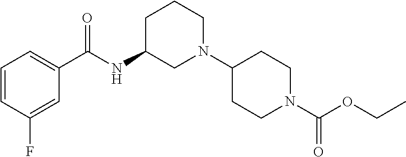 | >10 μM | (S)-ethyl 3-(3- fluorobenzamido)-1,4′- bipiperidine-1′-carboxylate | 378.3 |
| 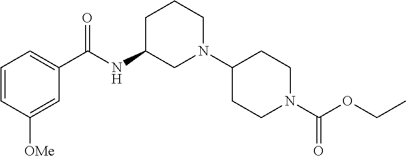 | >10 μM | (S)-ethyl 3-(3- methoxybenzamido)-1,4′- bipiperidine-1′-carboxylate | 390.2 |
| 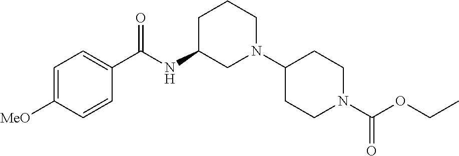 | >10 μM | (S)-ethyl 3-(4- methoxybenzamido)-1,4′- bipiperidine-1′-carboxylate | 390.2 |
| 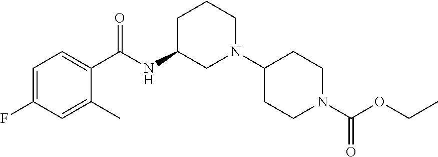 | >10 μM | (S)-ethyl 3-(4-fluoro-2- methylbenzamido)-1,4′- bipiperidine-1′-carboxylate | 392.2 |
| 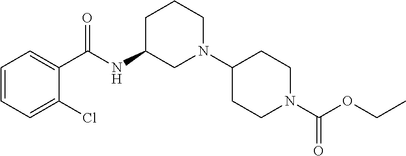 | >10 μM | (S)-ethyl 3-(2- chlorobenzamido)-1,4′- bipiperidine-1′-carboxylate | 394.2 |
| 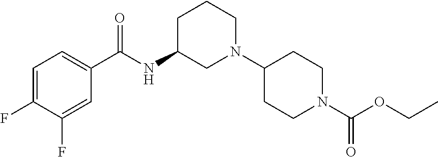 | >10 μM | (S)-ethyl 3-(3,4- difluorobenzarnido)-1,4′- bipiperidine-1′-carboxylate | 396.2 |

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 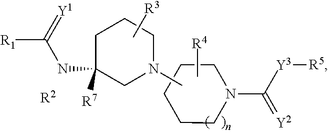
wherein n is an integer from 0 to 2;
wherein Y¹ and Y² are independently O or S;
wherein Y³ is a covalent bond, O, S, or N—R⁶;
wherein R¹ is an optionally substituted organic residue comprising from 1 to 12 carbons;
wherein R² is hydrogen, a hydrolysable residue, or an optionally substituted organic residue comprising 1 to 6 carbons;
wherein R³ comprises eight substituents independently selected from hydrogen, halogen, hydroxyl, nitrile, nitro, thiol, optionally substituted amino, and optionally substituted organic residue comprising from 1 to 6 carbons;
wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons;
wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl;
wherein R⁶, when present, is independently selected from hydrogen, a hydrolysable residue, and optionally substituted organic residue comprising from 1 to 6 carbons; and
wherein R⁷ is hydrogen or an optionally substituted organic residue comprising from 1 to 6 carbons,
or a pharmaceutically acceptable derivative thereof.

2. The compound of claim 1, wherein R¹ is selected from optionally substituted C1-C12 alkyl or C2-C12 alkenyl or C2-C12 alkynyl, optionally substituted C1-C12 heteroalkyl or C2-C12 heteroalkenyl or C2-C12 heteroalkynyl, optionally substituted C3-C12 cycloalkyl or C3-C12 cycloalkenyl, optionally substituted C3-C12 heterocycloalkyl or C3-C12 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl.

3. The compound of claim 1, wherein R² is hydrogen or a hydrolysable residue.

4. The compound of claim 1, wherein each R³ is independently selected from optionally substituted C1-C6 alkyl or C2-C6 alkenyl or C2-C6 alkynyl, optionally substituted C1-C6 heteroalkyl or C2-C6 heteroalkenyl or C2-C6 heteroalkynyl, optionally substituted C3-C6 cycloalkyl or C3-C6 cycloalkenyl, optionally substituted C3-C6 heterocycloalkyl or C3-C6 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl.

5. The compound of claim 1, wherein each R⁴ is independently selected from optionally substituted C1-C6 alkyl or C2-C6 alkenyl or C2-C6 alkynyl, optionally substituted C1-C6 heteroalkyl or C2-C6 heteroalkenyl or C2-C6 heteroalkynyl, optionally substituted C3-C6 cycloalkyl or C3-C6 cycloalkenyl, optionally substituted C3-C6 heterocycloalkyl or C3-C6 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl.

6. The compound of claim 1, wherein R⁵ is selected from optionally substituted C1-C12 alkyl or C2-C12 alkenyl or C2-C12 alkynyl, optionally substituted C1-C12 heteroalkyl or C2-C12 heteroalkenyl or C2-C12 heteroalkynyl, optionally substituted C3-C12 cycloalkyl or C3-C12 cycloalkenyl, optionally substituted C3-C12 heterocycloalkyl or C3-C12 heterocycloalkenyl, optionally substituted aryl, optionally substituted heteroaryl, optionally substituted alkoxyl, optionally substituted thioalkyl, optionally substituted alkylsulfinyl, optionally substituted alkylsulfonyl, optionally substituted alkylamino, thioamido, amidosulfonyl, alkoxycarbonyl, carboxamide, amino-carbonyl, and alkylamine-carbonyl.

7. The compound of claim 1, wherein n is 1; wherein Y¹═Y²═Y³═O; and wherein the compound has a structure represented by a formula: 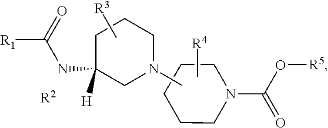
wherein R³ comprises eight substituents independently selected from hydrogen, halogen, hydroxyl, nitrile, nitro, thiol, optionally substituted amino, and optionally substituted organic residue comprising from 1 to 6 carbons, and
wherein R⁴ comprises nine substituents independently selected from hydrogen, halogen, hydroxyl, nitrile, nitro, thiol, optionally substituted amino, and optionally substituted organic residue comprising from 1 to 6 carbons.

8. The compound of claim 1, wherein n is 1; wherein Y¹═Y²═Y³═O; wherein all of R², R³, R⁴, and R⁷ are hydrogen; and wherein the compound has a structure represented by a formula:

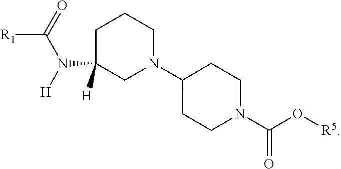

9. The compound of claim 1, wherein n is 1; wherein Y¹═Y²═Y³═O; wherein all of R², R³, R⁴, and R⁷ are hydrogen; and wherein the compound has a structure represented by a formula: 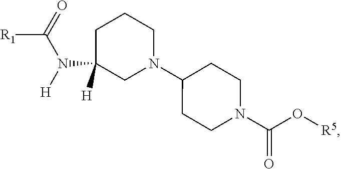
wherein R¹ is optionally substituted cycloalkyl, optionally substituted heterocycloalkyl, optionally substituted aryl, or optionally substituted heteroaryl;
wherein R⁵ is selected from optionally substituted C1-C6 alkyl; and
wherein the compound activates M₁ receptor response in M₁-transfected CHO-K1 cells, having an EC₅₀ of less than about 10 μM.

10. The compound of claim 9, wherein R¹ is selected from 2-chlorobenzene, 2-methylbenzene, 3,4-difluorobenzenem, 3-fluorobenzene, 3-methoxybenzene, 4-fluoro-2-methylbenzene, 4-methoxybenzene, 4-methylbenzene, cyclohexane, cyclopentane, phenyl, and thiophene.

11. The compound of claim 9, wherein R⁵ is ethyl.

12. A method for preparing a compound comprising the steps of:
a. providing an amino compound having a structure represented by a formula: 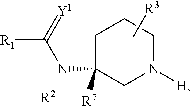
wherein Y¹ is O or S;
wherein R¹ is an optionally substituted organic residue comprising from 1 to 12 carbons;
wherein R² is hydrogen, a hydrolysable residue, or an optionally substituted organic residue comprising 1 to 6 carbons;
wherein R³ comprises eight substituents independently selected from hydrogen, halogen, hydroxyl, nitrile, nitro, thiol, optionally substituted amino, and optionally substituted organic residue comprising from 1 to 6 carbons;
wherein R⁷ is hydrogen or an optionally substituted organic residue comprising from 1 to 6 carbons, and
b. reacting the amino compound under reductive amination conditions with a cycloalkanone compound having a structure represented by a formula: 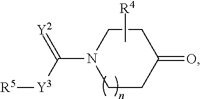
wherein n is an integer from 0 to 2;
wherein Y² is O or S;
wherein Y³ is a covalent bond, O, S, or N—R⁶;
wherein R⁴ comprises from six to ten substituents independently selected from hydrogen, halogen, hydroxyl, nitrile, nitro, thiol, optionally substituted amino, and optionally substituted organic residue comprising from 1 to 6 carbons;
wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl;
wherein R⁶, when present, is independently selected from hydrogen, a hydrolysable residue, and optionally substituted organic residue comprising from 1 to 6 carbons.

13. The method of claim 12, wherein the amino compound has a structure represented by a formula:

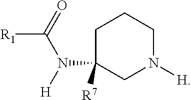

14. The method of claim 12, wherein the carboxyl compound has a structure represented by a formula:

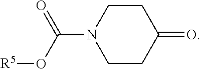

15. The method of claim 12, wherein providing comprises:
a. reacting an amino compound having a structure represented by a formula: 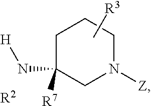
wherein R² is hydrogen, a hydrolysable residue, or an optionally substituted organic residue comprising 1 to 6 carbons;
wherein R³ comprises eight substituents independently selected from hydrogen, halogen, hydroxyl, nitrile, nitro, thiol, optionally substituted amino, and optionally substituted organic residue comprising from 1 to 6 carbons;
wherein R⁷ is hydrogen or an optionally substituted organic residue comprising from 1 to 6 carbons; and
Z is hydrogen or a protecting group,
with a carboxyl compound having a structure represented by a formula: 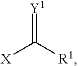
wherein Y¹ is O or S;
wherein R¹ is an optionally substituted organic residue comprising from 1 to 12 carbons; and
wherein X is a leaving group, and
b. optionally, removing the protecting group Z, when present.

16. The method of claim 12, wherein the compound formed has a structure represented by a formula:

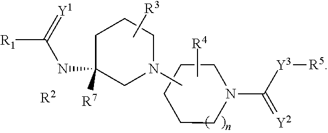

17. A method for the treatment of a disorder associated with selective M₁ receptor activation in a mammal comprising the step of administering to the mammal at least one compound having a structure represented by a formula: 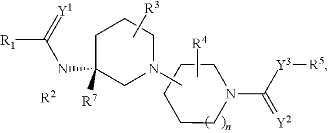
wherein n is an integer from 0 to 2;
wherein Y¹ and Y² are independently O or S;
wherein Y³ is a covalent bond, O, S, or N—R⁶;
wherein R¹ is an optionally substituted organic residue comprising from 1 to 12 carbons;
wherein R² is hydrogen, a hydrolysable residue, or an optionally substituted organic residue comprising 1 to 6 carbons;
wherein R³ comprises eight substituents independently selected from hydrogen, halogen, hydroxyl, nitrile, nitro, thiol, optionally substituted amino, and optionally substituted organic residue comprising from 1 to 6 carbons;
wherein R⁴ comprises from seven to eleven substituents independently selected from hydrogen, halogen, hydroxyl, nitrile, nitro, thiol, optionally substituted amino, and optionally substituted organic residue comprising from 1 to 6 carbons;
wherein R⁵ is hydrogen or an optionally substituted organic residue comprising from 1 to 12 carbons, with the proviso that wherein Y³ is a covalent bond, then R⁵ is hydrogen or optionally substituted C1-C6 alkyl;
wherein R⁶, when present, is independently selected from hydrogen, a hydrolysable residue, and optionally substituted organic residue comprising from 1 to 6 carbons; and
wherein R⁷ is hydrogen or an optionally substituted organic residue comprising from 1 to 6 carbons,
or a pharmaceutically acceptable derivative thereof, in a dosage and amount effective to treat the disorder in the mammal.

18. The method of claim 17, further comprising the step of identifying a subject in need of treatment for the disorder.

19. The method of claim 17, wherein the disorder is selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, conduct disorder, autistic disorder; movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

20. The method of claim 17, wherein the disorder is Alzheimer's disease.
